(19) United States
(12) Patent Application Publication
(10) Pub. No.: US 2009/0130154 A1
(43) Pub. Date: May 21, 2009

(54) Topical Delivery of Biological and Cosmetic Agents by Zeolites

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, SCOTTSDALE, AZ (US)

(21) Appl. No.: 12/361,615

(22) Filed: Jan. 29, 2009

Related U.S. Application Data
(63) Continuation-in-part of application No. 11/308,290, filed on Mar. 15, 2006.
Application No. 11/308,290 is a continuation-in-part of application No. 12/032,751, filed on Feb. 18, 2008, now abandoned.
Application No. 12/032,751 is a continuation-in-part of application No. 10/711,136, filed on Aug. 26, 2004, now abandoned.
Application No. 10/711,136 is a continuation-in-part of application No. 11/307,824, filed on Feb. 24, 2006.
Application No. 11/307,824 is a continuation-in-part of application No. 11/684,702, filed on Mar. 12, 2007, now abandoned.
Application No. 11/684,702 is a continuation-in-part of application No. 11/760,466, filed on Jun. 8, 2007.

(51) Int. Cl. A61K 8/02; A61Q 19/02; A61Q 19/08; A61Q 19/00; A61Q 5/00; A61Q 15/00

(52) U.S. Cl. 424/401

(57) ABSTRACT

The present invention discloses certain di- and polyvalent metal zeolite compounds (formula I) for topical delivery of biological and skin and hair care agents. The method of treating skin and hair condition via topical application of said zeolite compounds is also disclosed. The said method provides a treatment for topical condition, which includes alleviation of skin conditions such as skin rash including diaper rash, dry skin, scalp dandruff, broken or chafed skin, sunburn, skin damage from UV, skin irritation, acne including excess facial oil and facial pore size; darkened skin including age spots, dark circles around the eyes, and discoloration of skin from stretch marks; skin aging including wrinkles and fine lines; loss of collagen including thinning skin and loss of skin pliability; body odor, including oral cavity odor, arm-pit odor, and incontinence odor; cellular inflammation including intracellular and extra cellular inflammation; premature hair aging including premature hair loss hair graying; malfunction of tyrosinase group of enzymes, malfunction of matrix metalloprotease group of enzymes; and combinations thereof. The said method also provides topical delivery of certain metals, including trace metals, and certain zirconium aluminum amino acids that provide antiperspirant benefits;

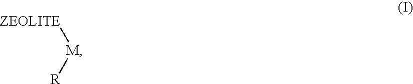

This is a continuation-in-part of U.S. patent application Ser. Nos. 11/308,290 (filed Mar. 15, 2006), 12/032,751 (filed Feb. 18, 2008), 10/711,136 (filed Aug. 26, 2004), 11/307,824 (filed Feb. 24, 2006), 11/684,702 (filed Mar. 12, 2007), and 11/760,466 (filed Jun. 8, 2007).

### BACKGROUND OF THE INVENTION

The present invention discloses certain di- or polyvalent metal zeolite compounds for topical delivery of biological and skin and hair care agents, and a method of treating skin and hair condition via topical application of said zeolite compounds. The said method provides a treatment for topical condition, which includes alleviation of skin conditions such as skin rash including diaper rash, dry skin, scalp dandruff, broken or chafed skin, sunburn, skin damage from UV, skin irritation, acne including excess facial oil and facial pore size; darkened skin including age spots, dark circles around the eyes, and discoloration of skin from stretch marks; skin aging including wrinkles and fine lines; loss of collagen including thinning skin and loss of skin pliability; body odor, including oral cavity odor, arm-pit odor, and incontinence odor; cellular inflammation including intracellular and extra cellular inflammation; premature hair aging including premature hair loss hair graying; malfunction of tyrosinase group of enzymes, malfunction of matrix metalloprotease group of enzymes; and combinations thereof. The said method also provides topical delivery of certain metals, including trace metals, and certain zirconium aluminum amino acids that provide antiperspirant benefits.

### DESCRIPTION OF THE RELATED ART

Zeolites are a group of crystalline aluminosilicates that have a porous, cage-like structure with a cavity. A zeolite may be defined as an aluminosilicate with a framework structure enclosing cavities occupied by large ions and water molecules, both of which have considerable freedom of movement, permitting ion-exchange and reversible dehydration. The framework consists of an open arrangement of corner sharing tetrahedral where SiO4 are partially replaced by AlO4 tetrahedra, which requires sufficient cations to achieve electro neutrality.

There are some 50 natural and over 150 synthetic zeolites, the latter all made by hydrothermal synthesis. The main uses are as molecular sieves, catalysts, and catalyst support for platinum group metals. Zeolite cavities are usually occupied by water.

Dehydration of synthetic zeolites leaves cubic micro crystals in which AlO4 and SiO4 tetrahedra are linked together to form a ring of eight O atoms on each face of the unit cube and an irregular ring of six O atoms across each corner. In the center of the unit cell is a large cavity about 11.4 Angstroms in diameter, which is connected to six identical cavities in adjacent unit cells by the eight-membered rings, which have inner diameter of about 4.2 Angstroms. In addition, the large cavity is connected to eight smaller cavities, about 6.6 Angstroms in diameter, by the six-membered rings, which provide openings of about 2.0 Angstrom in diameter. In the hydrated form all the cavities contain water molecules. In the anhydrous state the same cavities may be occupied by other molecules brought into contact with the zeolite, provided such molecules are able to pass through the apertures connecting cavities. Molecules within the cavities then tend to be held there by attractive forces of electrostatic and van der Waals types. Thus the zeolites will be able to absorb and strongly retain molecules just small enough to enter the cavities. It will not absorb at all those too big to enter. It will absorb weakly very small molecules that can enter or leave easily, except water molecules, which bind strongly.

The preparation and properties of anionic zeolites are described in detail in U.S. Pat. No. 2,882,243, among other sources. Generally, the preparation involves combining aqueous solutions that are sources of silica, alumina and sodium to produce a gel that crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300 C provides a zeolite that has a strong heat of hydration.

Zeolites can be made with both specific pore structures and bound cations such as Na, K, Mg, Ca, and Zn, that have found applications in various self-warming cosmetic compositions in the prior art. U.S. Pat. No. 3,250,680 (Menkart et al.) discloses applications of Zeolites for the preparation of self-heating toothpaste and other such compositions.

The inorganic complexes of zeolites are well known, for example, RU 2163510 discloses method for fixing metal complexes by the aid of T-shaped anchoring fragment in large cavities of zeolites.

U.S. patent application Ser. No. 2007000382 discloses silver trapped zeolite complexes.

DE 19913395 discloses certain inclusion compounds (I) based on a zeolite host lattice comprise metal clusters, transition metal complexes, noble metal complexes and dyes contained in mesopores of a zeolite, the mesopores being enclosed exclusively by micropores.

U.S. Pat. No. 5,429,814 disclose a method of using molecular sieve-enclosed paramagnetic ions as image brightening or image contrast agents. In particular, zeolite enclosed trivalent gadolinium is useful in MRI studies of the entire gastrointestinal tract.

U.S. Pat. No. 4,472,517 discloses a method of incorporating metals onto a crystalline aluminosilicate zeolite support comprises first depositing a metal which forms bonds with the zeolite and subsequently depositing a catalytically active metal into the zeolite which becomes associated with the first metal.

JP 2007197341 and JP2007196104 (Masuda et al.) disclose an antimicrobial agent that comprises zeolite, an oxygen-activating metal complex encapsulated in the unit cells of the zeolite, and a photo-reductive metal complex held in the zeolite (preferably encapsulated in the unit cells of the zeolite). Preferably, the zeolite contains a metal (for example, one or more metals selected from silver, copper and zinc) belonging to the group 1 to 13 in the periodic table. The oxygen-activating metal complex includes a metal phthalocyanine complex and a metal salophen complex synthesized in a unit cell by ship-in-bottle method. Masuda et al. do not disclose any organic non-antimicrobial agent complexes of zinc zeolites with said agents, and wherein said complexes are non-antimicrobial themselves but still impart topical benefits such as the treatment for acne that are not based on antimicrobial action of said complexes.

WO 02100420 discloses a pharmaceutical composition, which includes a synergic association of erythromycin and a carrier based on zeolite brought to Zn form. The preparation process is also described, which is based on an initial exchange of the Na ions present in the zeolite with Zn; this zeolite in zinc form is then mixed with erythromycin. However, it is should be noted that erythromycin, in itself, is well known to form complexes with many other molecules, for example, Mirza et al., “Influence of Solvents on the Variety of Crystalline Forms of Erythromycin”, AAPS Pharm. Sci. 2003; 5 (2): article 12, pages 1-9.

Azithromycin, a chemical structural analog of erythromycin, is also known to form compounds (U.S. Pat. Nos. 7,235,646; 6,245,903). It is thus not surprising or unexpected that erythromycin, similar to many other antibiotics, forms complexes with zeolites, including zinc zeolite. However, specific compounds of zinc zeolite with an antibiotic, for example zinc zeolite erythromycin or zinc zeolite azithromycin, have not been disclosed in the prior art.

U.S. Pat. No. 4,911,899 and U.S. Pat. No. 4,911,898 (Hagiwara et al.) disclose certain bactericidal zinc and silver zeolites, which do not include any non-antibacterial organic agents.

Benzaminson et al. (WO 2006098680) disclose the use of a hydrophobic zeolite, that contains an active component, especially a disinfection element, as ethanol, iodine, phenol, cresol or hydrogen peroxide, in a composition for non-medical treatment of the skin, for example as a deodorant. Benzaminson et al. also describe the use of a hydrophobic zeolite, that contains an active component, especially a disinfection element, as hydrogen peroxide, for manufacturing of a pharmaceutical preparation for treatment of skin related conditions and diseases, as skin infections. The hydrophobic zeolite is especially selected from that group that comprises silicalite, or hydrophobic ZMS-5, hydrophobic mordenite and hydrophobic zeolite Y.

U.S. Pat. No. 5,476,660 (Somasundaran et al.) discloses certain compositions of chemically modified zeolites in which zeolite surface has been modified to a positively charged state (cationic) or a zwitterionic state. These chemically modified zeolites have a filamentous structure with outwardly protruding positively charged organocarbonyl groups and also outwardly protruding negatively charged organocarbonyl groups. These chemically modified zeolites are useful for the deposition of active agents, more specifically, anionic active agents.

U.S. Pat. No. 4,626,550 (Hertzenberg) discloses certain personal care products such as lotions and creams that are prepared using potassium exchanged Zeolite A that is much less anionic in nature. These compositions are useful only for the release of heat, and the inclusion of active agents such as bodying agents, topical pain relievers, antiperspirants and others must be largely anhydrous and should not enter the structures of the zeolite to release heat (col. 3, line 50-57).

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various musculoskeletal problems. Varying the character of the liquid vehicle can control generation and maintenance of the heat of hydration of anhydrous zeolite. If a very quick release of heat is desired, a hydrophilic vehicle is used; if a slow, sustained heat release is desired, a hydrophobic vehicle is required. Intermediate and controlled performance can be introduced by altering the hydrophobic vehicle to provide some hydrophilic characteristics.

U.S. Pat. No. 6,274,128 (Bergman et al.) discloses an essentially anhydrous hair conditioning composition that comprises zeolites of specific pore size larger than the critical diameter of a water molecule and both the carrier molecules and the hair conditioner molecules that have molecular diameters larger than the largest average pore size of the micro porous materials.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect. Minnix utilizes only the heat-releasing or rubefacient properties of zeolites and does not disclose any zinc zeolite based compounds or a method of topical treatment with said compounds.

U.S. Application 20010016201 (Janchitraponvej) discloses a yet another self-heating application of an anhydrous rinse-out hair care composition utilizing zeolites.

Self-warming compositions have also been made with various anhydrous alkali metal salts (Giani et al., U.S. Pat. No. 5,747,004). In self-warming formulations based on Zeolites, the pore size specification is typically very small, from 3 to 10 angstroms in diameter, as is the ratio between sodium and potassium cations bound to silicate anions of such zeolites. These formulations release heat upon contact with water. Water penetrates the pores of such Zeolites and hydrates the interior silicate atoms of Zeolite agglomerates. Such interaction of zeolite with water releases the heat of hydration. Most cosmetic lotion, cream, shampoo, and conditioner products also contain hydrophilic and lipophilic ingredients for skin and hair care benefits. Some of such ingredients tend to clog the pores of Zeolites, causing a reduction in the heat-release properties of such formulations. The examples of such fatty materials that can inhibit the heat release properties of zeolites include most surfactants used in shampoo and body wash applications; quaternary ammonium compounds used for hair conditioning applications; fatty esters used as emollients in skin lotion and cream applications, and other similar examples. While such clogging of zeolite pores by above mentioned ingredients, some of which are highly desirable active agents, was considered a problem, and those problems were solved in the prior art by the use of small pore size zeolites that permit the entrance of water molecules inside their cavity but not other larger size molecules, for example U.S. Pat. No. 6,274,128.

U.S. patent application Ser. No. 20050133049 (Fournier et al.) discloses filters, smoking articles, and methods for selectively removing one or more selected constituents from mainstream smoke. The filters comprise zeolite BETA. Fournier et al. did discover that certain organic agents can bind with zeolite, but they did not disclose any zinc zeolite based compounds or a method of topical treatment with said compounds.

U.S. patent application Ser. No. 20050058597 (Corbin et al.) discloses a process to synthesize nano-size Zeolite A from an amorphous gel precursor. The nano-sized Zeolite A has been used for detergents. Corbin et al. did not disclose any zinc zeolite based compounds or a method of topical treatment with said compounds.

It is worthy of note that although zeolites with many different cations, such as titanium, zinc, manganese, iron, and copper have been disclosed, any applications of such metal zeolites in any zinc zeolite based biological or skin or hair care compound or a method of topical treatment with said compound has not been disclosed in the prior art. Additional pertinent prior art examples include Mangione et al. (U.S. Pat. No. 6,485,714), Pilleux et al. (U.S. Pat. No. 5,346,693), Painter et al. (U.S. Pat. No. 6,117,435), Barker et al. (U.S. Pat. No. 4,177,259, and Carr et al. (EP 0739388). However, none of these prior art references disclose any di- or polyvalent metal zeolite compounds.

U.S. Pat. No. 6,503,740 (Alther et al.) discloses zeolites treated with an organic modification compound such as quaternary amines, pyridinium compounds, and phosphonium amines that are useful for water treatment applications. Alther et al. do not disclose any zinc zeolite based compounds or a method of topical treatment with said compounds.

U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman). However, none of these disclose any zinc zeolite based compounds or a method of topical treatment with said compounds.

EP 1683761 discloses the preparation of zeolite ZBM-30, which is a mixture of a zeolite, an amine, and a tri- or tetravalent metal.

Tanimoto et al. (U.S. Pat. No. 6,071,542) disclose a number of metal zeolites with antibacterial activity. These are unlike the metal zeolite compounds of the present invention.

### BRIEF SUMMARY OF THE INVENTION

The present invention discloses certain di- and polyvalent metal zeolite compounds as topical delivery systems for biological and skin and hair care agents. These compounds are not mere encapsulations, micro encapsulations, or entrapments of organic or inorganic molecules within a zeolite cavity.

The present invention also discloses a method of treating skin and hair condition via topical application of said zeolite compounds. The said method provides a treatment for topical condition, which includes alleviation of skin conditions such as skin rash including diaper rash, dry skin, scalp dandruff, broken or chafed skin, sunburn, skin damage from UV, skin irritation, acne including excess facial oil and facial pore size; darkened skin including age spots, dark circles around the eyes, and discoloration of skin from stretch marks; skin aging including wrinkles and fine lines; loss of collagen including thinning skin and loss of skin pliability; cellular inflammation including intracellular and extra cellular inflammation; premature hair aging including premature hair loss hair graying; malfunction of tyrosinase group of enzymes, malfunction of matrix metalloprotease group of enzymes; and combinations thereof.

### DETAILED DESCRIPTION

The present invention discloses a di- or polyvalent metal zeolite compound (zeolite compound) of formula (I) for topical application;

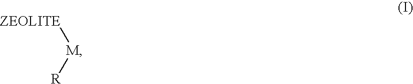

Wherein,
Zeolite is selected from a group of synthetic zeolites consisting of zeolite, pore size 3A; zeolite pore size 4A; zeolite, pore size 5A; zeolite, pore size 9A; zeolite, pore size 13×; calcium zeolite; lithium zeolite; high silica zeolite, and other similar zeolites with controlled pore sizes of from 3 Angstroms to 30 Angstroms.
M is selected from Zn, Mn, Cu, Mo, Ti, Fe, Ni, Cr, Co, V, Ca, Ba, Mg, Se, and Al; and
R is selected from O-alkyl, O-cycloalkyl, O-aralkyl, O-aryl, O-heterocyclic, O-vinyl, O-vinyl alkyl, O-vinyl aryl, O-vinyl heterocyclic, O-keto alkyl, O-keto aryl, O-keto heterocyclic, O-keto peptide, O-keto alkyl amine, O-phosphate ester, O-carboxy alkyl, O-carboxy aryl, S-alkyl, S-cycloalkyl, S-aralkyl, S-aryl, S-heterocyclic, S-vinyl, S-vinyl alkyl, S-vinyl aryl, S-vinyl heterocyclic, S-keto alkyl, S-keto aryl, S-keto heterocyclic, S-keto alkyl amine, and zirconium aluminum amino acid.

The present invention also discloses a method for treatment of skin or hair condition, wherein (i) said zeolite compound is applied topically at a desired site in a sufficient quantity, and (ii) wherein said application having been done either by a manual or a mechanical mean, or a combination thereof, and (iii) wherein said topical application causes the desired treatment of said skin or hair condition.

In the present invention, said skin or hair condition is selected from the group consisting of skin rash including diaper rash, dry skin, scalp dandruff, broken or chafed skin, sunburn, skin damage from UV, skin irritation, acne including excess facial oil and facial pore size; darkened skin including age spots, dark circles around the eyes, and discoloration of skin from stretch marks; skin aging including wrinkles and fine lines; loss of collagen including thinning skin and loss of skin pliability; cellular inflammation including intracellular and extra cellular inflammation; premature hair aging including premature hair loss hair graying; malfunction of tyrosinase group of enzymes, malfunction of matrix metalloprotease group of enzymes; and combinations thereof.

In the present invention, said O-phosphate ester substituent; R in (I), has a large number of possibilities, some of which include thiamine diphosphate substituent, (II); thiamine triphosphate substituent, (III); adenosine diphosphate substituent, (IV); adenosine triphosphate substituent, (V); and benfotiamine substituent, (VI):

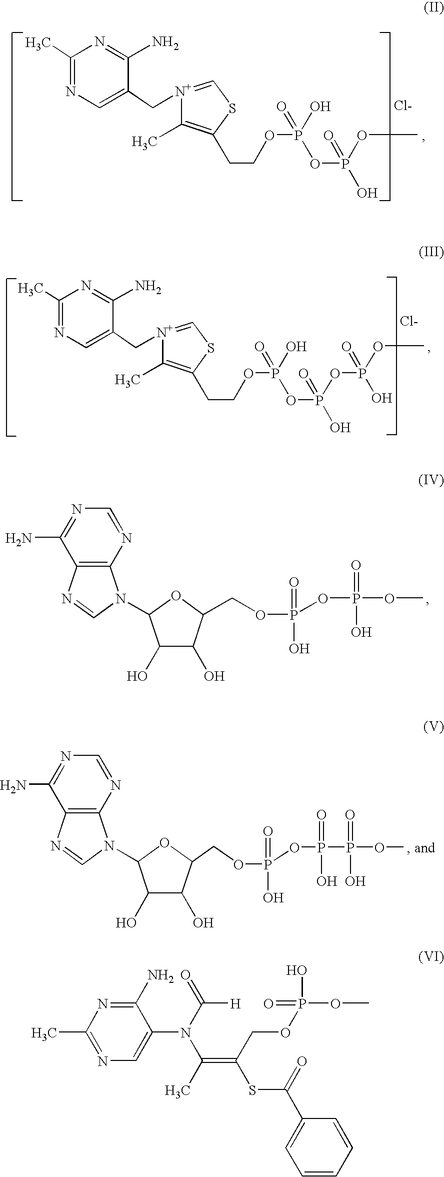

In the present invention, said O-alkyl, O-aryl, and O-heterocyclic substituents, which can also be a substituted alkyl or a substituted aryl; R in (I), have a large number of possibilities, some of which, for example, include thiamine substituent, (VII); retinal substituent, (VIII); calciferol substituent, (IX); tocopherol substituent, (X); and 8-hydroxyquinoline substituent, (XI):

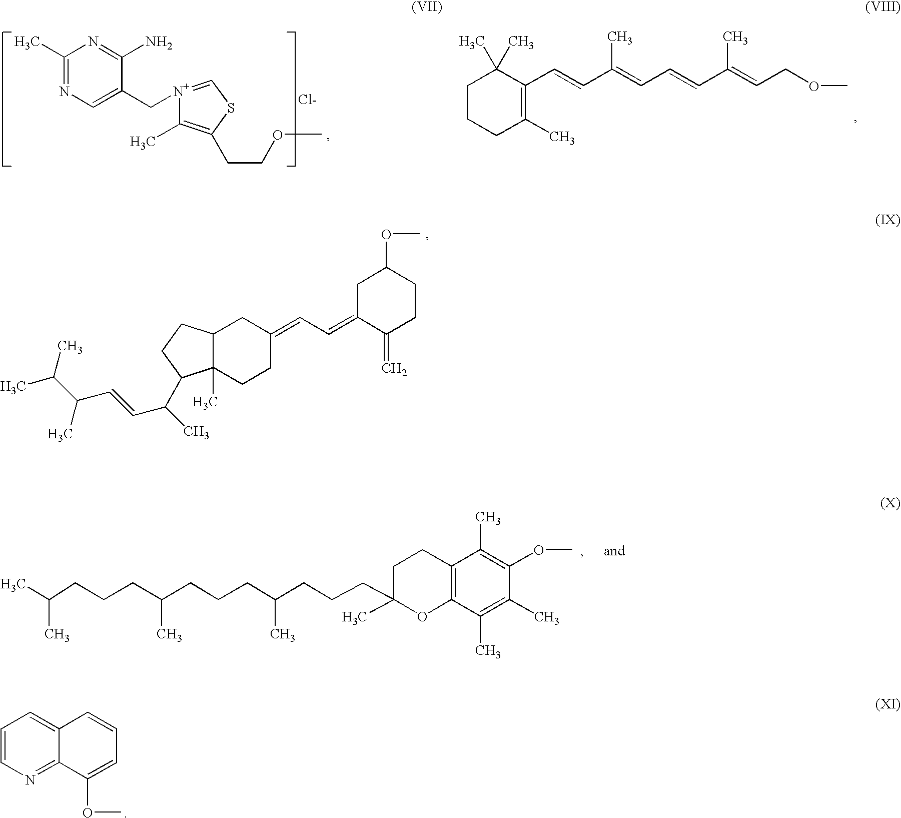

In the present invention, said O-keto alkyl, O-keto aryl, and O-keto heterocyclic substituents, which can also be a substituted O-keto alkyl, or a substituted O-keto aryl, or a substituted O-keto heterocyclic; R in (I), have a large number of possibilities, some of which, for example, include ascorbate, (XII); gluconate, (XIII); lactate, (XIV); salicylate, (XV); and pyridine carboxylate (XVI):

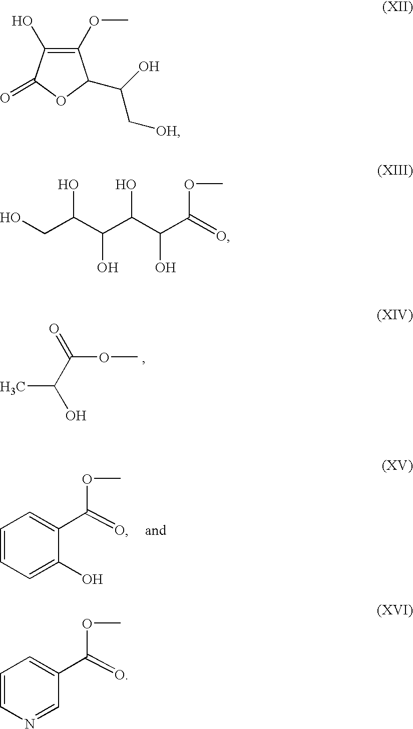

In the present invention, said O-keto alkyl amine, O-carboxy alkyl, O-carboxy aryl, and O-keto peptide substituents are selected from a large number of possibilities, some of which, for example, include peroxy benzoyl, (XVII); alkyl amino acyl, (XVIII), peptide carboxyl (carnosyl), (XIX), and S-alkyl (glutathionate), (XX):

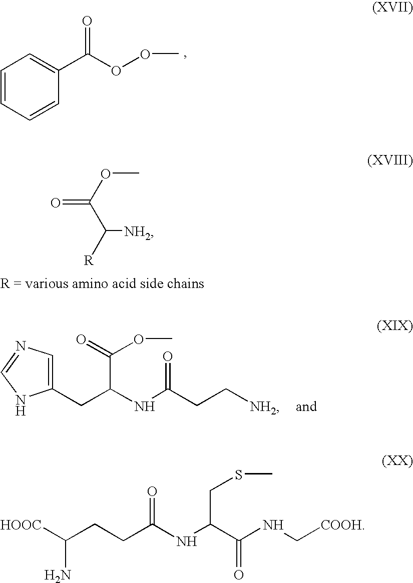

A large number of zeolite compounds are thus possible by the present invention, representative examples of which include zinc zeolite salicylate, (XXI); zinc zeolite lactate, (XXII); zinc zeolite ethylhexylglycerin, (XXIII); zinc zeolite benzoyl peroxide, (XXIV); copper zeolite adenosine triphosphate, (XXV); chromium zeolite benfotiamine, (XXVI); and oxo vanadium zeolite pyridoxal-5-phosphate, (XXVII):

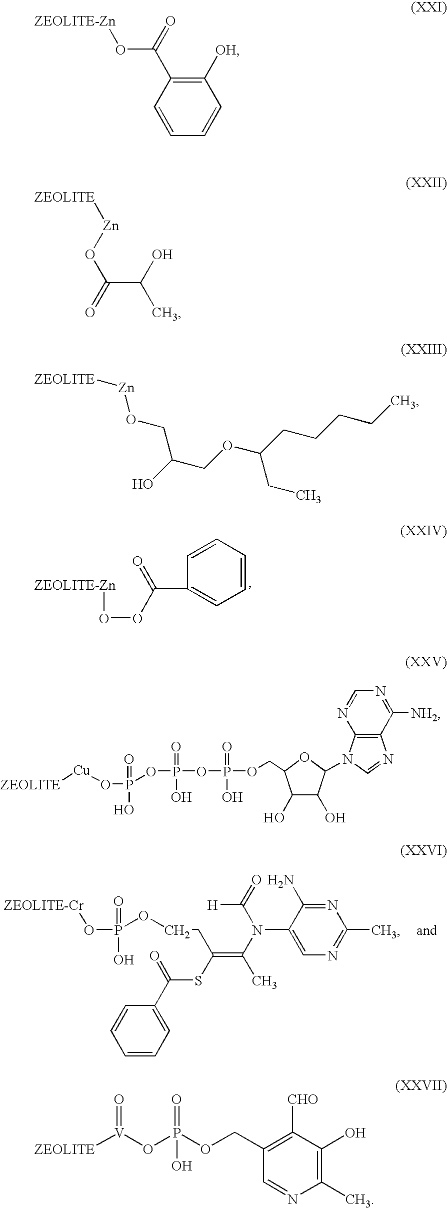

In the present invention, said compounds of metal zeolite hydroxy acids, their esters, and salts are further selected from, but not limited to alpha, beta, and polyhydroxy acids, their esters and salts.

The metal zeolite hydroxy acids and their salts are further selected from, but not limited to, metal zeolite compounds of 2-hydroxyethanoic acid (glycolic acid), 2-hydroxypropanoic acid (lactic acid), 2-methyl 2-hydroxypropanoic acid (methyl lactic acid), 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxyeicosanoic acid (alpha hydroxyarachidonic acid), 2-hydroxytetraeicosanoic acid (cerebronic acid), 2-hydroxytetraeicosenoic acid (alpha hydroxynervonic acid), 2,4-dihydroxy-3,3-dimethylbutanoic acid (pantoic acid), 2-phenyl 2-hydroxyethanoic acid (mandelic acid); 2,2-diphenyl 2-hydroxyethanoic acid (benzilic acid), 3-phenyl 2-hydroxypropanoic acid (phenyllacetic acid), 2-phenyl 2-methyl 2-hydroxyethanoic acid (atrolactic acid), 4-hydroxymandelic acid, 2,3-dihydroxypropanoic acid (glyceric acid); 2,3,4-trihydroxybutanoic acid (isomers; erythronic acid, threonic acid); 2,3,4,5-tetrahydroxypentanoic acid (isomers; ribonic acid, arabinoic acid, xylonic acid, lyxonic acid); 2,3,4,5,6-pentahydroxyhexanoic acid (isomers; allonic acid, altronic acid, gluconic acid, mannoic acid, gulonic acid, idonic acid, galactonic acid, talonic acid); 2,3,4,5,6,7-hexahydroxyheptanoic acid, 2-hydroxypropane-1,3-dioic acid (tartronic acid); 2-hydroxybutane-1,4-dioic acid (malic acid); 2-hydroxy-2-methylbutane-1,4-dioic acid (citramalic acid); 2,3-dihydroxybutane-1,4-dioic acid (tartaric acid); 2,3,4-trihydroxypentane-1,5-dioic acid (isomers; ribaric acid, arabaric acid, xylaric acid, lyxaric acid); 2,3,4,5-tetrahydroxyhexane-, 1,6-dioic acid (isomers; glucaric acid, galactaric acid, mannaric acid, allaric acid, altraric acid, gularic acid, idaric acid, talaric acid); 2-hydroxy-1,2,3-propanetricarboxylic acid (citric acid); Hydroxycitric acid, Garcinia Acid, 1-hydroxy-1,2,3-propanetricarboxylic acid (isocitric acid); 1-hydroxy-1,2,4-butanetricarboxylic acid (homoisocitric acid); 2-hydroxy-3-hexadecyl-1,2,3-propanetricarboxylic acid, glyceruronic acid, erythruronic acid, threuronic acid; 2,3,4-trihydroxypentanuronic acids (isomers; riburonic acid, arabinuronic acid, xyluronic acid, lyxuronic acid); 2,3,4,5-tetrahydroxyhexanuronic acid (isomers; alluronic acid, altruronic acid, glucuronic acid, mannuronic acid, guluronic acid, iduronic acid, galacturonic acid, taluronic acid), and 2,3,4,5,6-pentahydroxyheptanuronic acid (isomers; alloheptanuronic acid, altroheptanuronic acid, glucoheptanuronic acid, mannoheptanuronic acid, guloheptanuronic acid, idoheptanuronic acid, galactoheptanuronic acid, taloheptanuronic acid, and the corresponding zinc salts of these acids, and combinations thereof.

The metal zeolite beta hydroxy acids and their salts are selected from salicylic acid and zinc salicylate.

The compounds of metal zeolite hydroxy esters are selected from, but not limited to zinc zeolite compounds of an ester of a hydroxy acid selected from the group consisting of:

the methyl, ethyl, propyl, isopropyl, butyl, pentyl, octyl, decyl and dodecyl esters of glycolic acid;
the methyl, ethyl, propyl, isopropyl, butyl, pentyl, octyl, decyl and dodecyl esters of lactic acid;
the methyl, ethyl, propyl, isopropyl, butyl, pentyl, octyl, decyl and dodecyl esters of methyl lactic acid;
the methyl, ethyl, propyl, isopropyl, butyl, pentyl, octyl, decyl and dodecyl esters of 2-hydroxybutanoic acid;
the methyl, ethyl, propyl, isopropyl, butyl, pentyl, octyl, decyl and dodecyl esters of 2-hydroxypentanoic acid; the methyl, ethyl, propyl, isopropyl, butyl, pentyl, octyl, decyl and dodecyl esters of 2-hydroxyhexanoic acid;
the methyl, ethyl, propyl, isopropyl, butyl, pentyl, octyl, decyl and dodecyl esters of 2-hydroxyheptanoic acid; the methyl, ethyl, propyl, isopropyl, butyl, pentyl, octyl, decyl and dodecyl esters of 2-hydroxyoctanoic acid;
the methyl, ethyl, propyl, isopropyl, butyl, pentyl, octyl, decyl and dodecyl esters of 2-hydroxynonanoic acid;
the methyl, ethyl, propyl, isopropyl, butyl, pentyl, octyl, decyl and dodecyl esters of 2-hydroxydecanoic acid;
the methyl, ethyl, propyl, isopropyl, butyl, pentyl, octyl, decyl and dodecyl esters of 2-hydroxyundecanoic acid;
the methyl, ethyl, propyl, isopropyl, butyl, pentyl, octyl, decyl and dodecyl esters of 2-hydroxydodecanoic acid;
the methyl, ethyl, propyl, isopropyl, butyl, pentyl, octyl, decyl and dodecyl esters of 2-hydroxytetradecanoic acid;
the methyl, ethyl, propyl, isopropyl, butyl, pentyl, octyl, decyl and dodecyl esters of 2-hydroxyhexadecanoic acid;
the methyl, ethyl, propyl, isopropyl, butyl, pentyl, octyl, decyl and dodecyl esters of 2-hydroxyoctadecanoic acid;
the methyl, ethyl, propyl, isopropyl, butyl, pentyl, octyl, decyl and dodecyl esters of 2-hydroxyeicosanoic acid;
the methyl, ethyl, propyl, isopropyl, butyl, pentyl, octyl, decyl and dodecyl esters of 2-hydroxytetraeicosanoic acid; and
the methyl, ethyl, propyl, isopropyl, butyl, pentyl, octyl, decyl and dodecyl esters of 2-hydroxytetraeicosenoic acid, the corresponding acids of these esters, and the corresponding zinc salts of those acids, and combinations thereof.

The zinc zeolite poly hydroxy acids, their esters, and salts are selected from compounds of zinc zeolite and any of the methyl, ethyl, propyl, isopropyl, butyl, pentyl, octyl, decyl and dodecyl esters of 2-phenyl 2-hydroxyethanoic acid esters;

the methyl, ethyl, propyl, isopropyl, butyl, pentyl, octyl, decyl and dodecyl esters of 2,2-diphenyl 2-hydroxyethanoic acid;
the methyl, ethyl, propyl, isopropyl, butyl, pentyl, octyl, decyl and dodecyl esters of 3-phenyl 2-hydroxypropanoic acid; and
the methyl, ethyl, propyl, isopropyl, butyl, pentyl, octyl, decyl and dodecyl esters of 2-phenyl 2-methyl 2-hydroxyethanoic acid, the methyl, ethyl, propyl, isopropyl, butyl, pentyl, octyl, decyl, dodecyl, phenyl and benzyl esters of 2,3-dihydroxypropanoic acid;
the methyl, ethyl, propyl, isopropyl, butyl, pentyl, octyl, decyl, dodecyl, phenyl and benzyl esters of 2,3,4-trihydroxybutanoic acid and its isomers including erythronic acid and threonic acid; the methyl, ethyl, propyl, isopropyl, butyl, pentyl, octyl, decyl, dodecyl, phenyl and benzyl esters of 2,3,4,5-tetrahydroxypentanoic acid and its isomers including ribonic acid, arabinoic acid, xylonic acid and lyxonic acid;
the methyl, ethyl, propyl, isopropyl, butyl, pentyl, octyl, decyl, dodecyl, phenyl and benzyl esters of 2,3,4,5,6-pentahydroxyhexanoic acid and its isomers including allonic acid, altronic acid, gluconic acid, mannoic acid, gulonic acid, idonic acid, galactonic acid, and talonic acid; the methyl, ethyl, propyl, isopropyl, butyl, pentyl, octyl, decyl, dodecyl, phenyl and benzyl esters of 2,3,4,5,6,7-hexahydroxyheptanoic acid and its isomers including glucoheptonic acid and galactoheptonic acid;
the methyl, ethyl, propyl, isopropyl, butyl, pentyl, octyl, decyl, dodecyl, phenyl and benzyl esters of glyceruronic acid;
the methyl, ethyl, propyl, isopropyl, butyl, pentyl, octyl, decyl, dodecyl, phenyl and benzyl esters of erythruronic acid;
the methyl, ethyl, propyl, isopropyl, butyl, pentyl, octyl, decyl, dodecyl, phenyl and benzyl esters of threuronic acid;
the methyl, ethyl, propyl, isopropyl, butyl, pentyl, octyl, decyl, dodecyl, phenyl and benzyl esters of riburonic acid;
the methyl, ethyl, propyl, isopropyl, butyl, pentyl, octyl, decyl, dodecyl, phenyl and benzyl esters of arabinuronic acid;
the methyl, ethyl, propyl, isopropyl, butyl, pentyl, octyl, decyl, dodecyl, phenyl and benzyl esters of xyluronic acid;
the methyl, ethyl, propyl, isopropyl, butyl, pentyl, octyl, decyl, dodecyl, phenyl and benzyl esters of lyxuronic acid;
the methyl, ethyl, propyl, isopropyl, butyl, pentyl, octyl, decyl, dodecyl, phenyl and benzyl esters of alluronic acid;
the methyl, ethyl, propyl, isopropyl, butyl, pentyl, octyl, decyl, dodecyl, phenyl and benzyl esters of altruronic acid;
the methyl, ethyl, propyl, isopropyl, butyl, pentyl, octyl, decyl, dodecyl, phenyl and benzyl esters of glucuronic acid;
the methyl, ethyl, propyl, isopropyl, butyl, pentyl, octyl, decyl, dodecyl, phenyl and benzyl esters of mannuronic acid;
the methyl, ethyl, propyl, isopropyl, butyl, pentyl, octyl, decyl, dodecyl, phenyl and benzyl esters of guluronic acid;
the methyl, ethyl, propyl, isopropyl, butyl, pentyl, octyl, decyl, dodecyl, phenyl and benzyl esters of iduronic acid;
the methyl, ethyl, propyl, isopropyl, butyl, pentyl, octyl, decyl, dodecyl, phenyl and benzyl esters of galacturonic acid; and
the methyl, ethyl, propyl, isopropyl, butyl, pentyl, octyl, decyl, dodecyl, phenyl and benzyl esters of taluronic acid, and combinations thereof.

In the present invention, said compounds of metal zeolite organic peroxides and hydroperoxides are further selected from, but not limited to metal zeolite benzoyl peroxide, metal zeolite artemisinin, metal zeolite artemisia annua extract, and metal zeolite benzoyl hydroperoxide.

In the present invention, said compounds of metal zeolite lignans are further selected from, but not limited to metal zeolite silybin, metal zeolite silymarin, metal zeolite silydianin, metal zeolite silychristin, metal zeolite isosilybin, metal zeolite sauriol, metal zeolite licarin, metal zeolite saucernetin, metal zeolite saucerneol, metal zeolite niranthin, metal zeolite Phyllanthin, metal zeolite manassantins, metal zeolite matairesinol, metal zeolite hydroxymatairesinol, metal zeolite oxomatairesinol, metal zeolite saminol, metal zeolite americanin, metal zeolite arctiin, metal zeolite arctigenin, metal zeolite lariciresinol, metal zeolite isolariciresinol, metal zeolite secoisolariciresinol, metal zeolite secoisolariciresinol diglycoside, metal zeolite rubrisandrin, metal zeolite egonol, metal zeolite masutakeside, metal zeolite styraxlignolide, metal zeolite lappaol, metal zeolite diarctigenin, metal zeolite interiotherin, metal zeolite schisandrol, metal zeolite schisandrin, metal zeolite sesamin, metal zeolite sesaminol, metal zeolite episesamin, metal zeolite episesaminol, metal zeolite sesamolin, metal zeolite verbascoside, metal zeolite tetrahydrocurcumin, metal zeolite rosmarinic acid, metal zeolite chlorogenic acid, metal zeolite guaiaretic acid, metal zeolite dihydroguiaretic acid, metal zeolite nor-dihydroguiaretic acid, metal zeolite alpha-conidendrin, metal zeolite liovil, metal zeolite picearesinol, metal zeolite syringaresinol, metal zeolite nortrachelogenin; their analogs and derivatives, and combinations thereof.

In the present invention, said compounds of metal zeolite saponins and sapogenins are further selected from, but not limited to metal zeolite Dioscin, metal zeolite Diosgenin, metal zeolite Hecogenin, metal zeolite Heconin, metal zeolite Tigogenin, metal zeolite Tigonin, metal zeolite Gitogenin, metal zeolite Chlorogenin, metal zeolite Eruboside, metal zeolite Protoeruboside, metal zeolite Manogenin, metal zeolite Shlorogenin, metal zeolite Hainangenin, metal zeolite Protodioscin, metal zeolite Protodiosgenin, metal zeolite Aculeoside, metal zeolite Smilagenin, metal zeolite Sarsapogenin, metal zeolite Yamogenin, metal zeolite Yuccagenin, metal zeolite Gracillin, metal zeolite Sativoside, and combinations thereof.

In present invention, said compounds of metal zeolite enzyme inhibitor includes metal zeolite matrix metalloprotease inhibitor, metal zeolite tyrosinase inhibitor, metal zeolite superoxide dismutase inhibitor, metal zeolite 5-alpha reductase inhibitor, metal zeolite Tumor Necrosis Factor (TNF)-alpha inhibitor, metal zeolite Ubiquitin—Proteasome inhibitor, metal zeolite advanced glycation end product inhibitor, metal zeolite citrate lyase inhibitor, metal zeolite fatty acid desaturase inhibitor, metal zeolite urocanate inhibitor, and metal zeolite prostaglandin—leukotriene pathway inhibitor, and combinations thereof.

In the present invention, said compounds of metal zeolite matrix metalloprotease inhibitors are further selected from, but not limited to metal zeolite 2-hydroxyacetophenone, metal zeolite 3-hydroxyacetophenone, metal zeolite 4-hydroxyacetophenone, metal zeolite 2,3-dihydroxyacetophenone, metal zeolite 3,4-dihydroxyacetophenone, metal zeolite 3,5-dihydroxyacetophenone, metal zeolite 2,4,6-trihydroxyacetophenone, metal zeolite 2,3,4-trihydroxyacetophenone, metal zeolite 2,3,5-trihydroxyacetophenone, metal zeolite 2,3,6-trihydroxyacetophenone, metal zeolite 2,4,5-trihydroxyacetophenone, metal zeolite 3,4,5-trihydroxyacetophenone, metal zeolite Resacetophenone, metal zeolite Quinacetophenone, metal zeolite 1-(3-Hydroxy-4-methoxy-5-methylphenyl)ethanone, metal zeolite 1-(3-hydroxy-4-methoxyphenyl)ethanone, metal zeolite Paeonol, metal zeolite 5′-Bromo-2′-hydroxyacetophenone, metal zeolite 5′-Chloro-2′-hydroxyacetophenone, metal zeolite 3′,5′-Dichloro-2′-hydroxyacetophenone, metal zeolite 3′,5′-Dibromo-4′-hydroxyacetophenone, metal zeolite 5-Chloro-3-bromo-2-hydroxyacetophenone, metal zeolite 2-hydroxypropiophenone, metal zeolite 3-hydroxypropiophenone, metal zeolite 4-hydroxypropiophenone, metal zeolite 2,3-dihydroxypropiophenone, metal zeolite 2,4-dihydroxypropiophenone, metal zeolite 2,5-dihydroxypropiophenone, metal zeolite 2,6-dihydroxypropiophenone, metal zeolite 3,4-dihydroxypropiophenone, metal zeolite 3,5-dihydroxypropiophenone, metal zeolite 2,4,6-trihydroxypropiophenone, metal zeolite 2,3,4-trihydroxypropiophenone, metal zeolite 2,3,5trihydroxypropiophenone, metal zeolite 2,3,6-trihydroxypropiophenone, metal zeolite 2,4,5-trihydroxypropiophenone, metal zeolite 3,4,5-trihydroxypropiophenone, metal zeolite 1-(2,4-dihydroxyphenyl)-2-hydroxyethanone, metal zeolite (2-hydroxyphenyl)(oxo)acetic acid, metal zeolite 1-(2,6-dihydroxyphenyl)-1-butanone, metal zeolite 1-(1-hydroxy-2-naphthyl)ethanone, metal zeolite 1-(2-hydroxy-1-naphthyl)ethanone, metal zeolite 5,7-dihydroxy-1-indanone, metal zeolite 1-(2-hydroxy-5-methylphenyl)-1,3-butanedione, metal zeolite N-(4-acetyl-3-hydroxyphenyl)acetamide, metal zeolite 4-acetyl-3-hydroxyphenyl acetate, metal zeolite 1,1′-(4,6-Dihydroxy-1,3-phenylene)bisethanone, metal zeolite 1-(1-hydroxy-2-naphthyl)ethanone, metal zeolite 2,3-Dihydro-9,10-dihydroxy-1,4-anthracenedione, metal zeolite phloridzin, metal zeolite phloretin, and combinations thereof.

In the present invention, said compounds of metal zeolite citrate lyase inhibitors are further selected from, but not limited to metal zeolite compounds of hydroxycitric acid and zinc hydroxycitrate.

In the present invention, metal zeolite tyrosinase inhibitors are further selected from, but not limited to metal zeolite hydroquinone, metal zeolite arbutin, metal zeolite kojic acid, metal zeolite hydroquinone derivatives, metal zeolite Paper Mulberry extract (Broussonetia kazinoke), metal zeolite Mitracarpe extract (Mitracarpus scaber), metal zeolite Bearberry extract (Arctostaphylos uva ursi), metal zeolite Yellow Dock extract (Rumex crispus and Rumex occidentalis), metal zeolite Glutathione, metal zeolite Leucocyte extract, metal zeolite Aspergillus orizae extract (Aspergillus orizae), metal zeolite Licorice Root extract (Glycyrrhiza glabra), metal zeolite Rosmarinic acid (Rosmarinus officinalis), metal zeolite Tetrahydrocurcumin, metal zeolite Green Tea extract (Camellia sinensis), metal zeolite Yohimbe extract (Pausinystalia yohimbe), metal zeolite Ecklonia cava extract, metal zeolite niacinamide, metal zeolite Hydroxytetronic acid, metal zeolite Spondias mombin extract, metal zeolite Maprounea guianensis extract, metal zeolite Walteria indica extract, metal zeolite Gouania blanchetiana extract, metal zeolite Cordia schomburgkii extract, metal zeolite Randia armata extract, metal zeolite Hibiscus furcellatus extract, and combinations thereof.

In the present invention, metal zeolite triterpenes are further selected from, but not limited to metal zeolite Asiaticoside (Centella asiatica extract), metal zeolite Boswellia Extract (Boswellia serrata), metal zeolite Sericoside, metal zeolite Visnadine, metal zeolite Thiocolchicoside, metal zeolite Glycyrrhetinic acid, metal zeolite Ursolic acid, metal zeolite Sericoside (Terminalia sericea extract), metal zeolite Darutoside (Siegesbeckia orientalis extract), and combinations thereof.

In the present invention, metal zeolite sunscreen agents are further selected from, but not limited to metal zeolite Kaempferia galanga extract, metal zeolite para-Aminobenzoic acid (PABA), metal zeolite Avobenzone, metal zeolite 3-Benzylidene camphor, metal zeolite Benzylidene camphor sulfonic acid, metal zeolite Bisymydazilate, metal zeolite Camphor Benzalkonium Methosulfate, metal zeolite Polyquaternium-59, metal zeolite Cinnamidipropyltrimonium chloride, metal zeolite Diethylamino hydroxybenzoyl hexyl benzoate, metal zeolite Diethylhexyl butamido triazone, metal zeolite Dimethicodiethylbenzal malonate, metal zeolite Drometrizole trisiloxane, metal zeolite Ecamsule, metal zeolite Ensulizole, metal zeolite Homosalate, metal zeolite Isoamyl p-methoxycinnamate, metal zeolite 4-Methylbenzylidene camphor, metal zeolite Octocrylene, metal zeolite Octyl Dimethyl PABA, metal zeolite Cinoxate, metal zeolite Dioxybenzone, metal zeolite Octyl methoxycinnamate, metal zeolite Octyl salicylate, metal zeolite Octyl triazone, metal zeolite Oxybenzone, metal zeolite Polyethylene glycol (PEG)-25 PABA, metal zeolite Polyacrylamidomethyl benzylidene camphor, metal zeolite Sulisobenzone, metal zeolite Methyl anthranilate, metal zeolite Trolamine salicylate, metal zeolite Benzophenone-3, metal zeolite Benzophenone-4, metal zeolite Tinosorb M (metal zeolite methylene bis benzotriazolyl tetramethylbutylphenol), metal zeolite Tinosorb S (metal zeolite Bemotrizinol), and combinations thereof.

In the present invention, metal zeolite stilbenes are further selected from, but not limited to metal zeolite rhapontin, metal zeolite polydatin, metal zeolite resveratrol, and combinations thereof.

In the present invention, metal zeolite peroxides are further elected from, but not limited to metal zeolite urea peroxide, metal zeolite urea hydrogen peroxide, metal zeolite perbenzoate, metal zeolite peracetate, metal zeolite meta-chloroperbenzoate, metal zeolite 2-butanone peroxide, metal zeolite tert-amyl peroxide, metal zeolite tert-butyl peroxide, metal zeolite cumyl peroxide, metal zeolite lauroyl peroxide, metal zeolite 2,4-Pentanedione peroxide, metal zeolite percarbonate, metal zeolite persulfate, and/or combinations thereof.

In the present invention, said compounds of metal zeolite polyphenols are further selected from, but not limited to metal zeolite magnolol, metal zeolite honokiol, metal zeolite ellagic acid, metal zeolite hypericin, metal zeolite tetrahydrocurcumin, metal zeolite mulberrin, metal zeolite rosmarinic acid, metal zeolite chlorogenic acid, metal zeolite licoricidin, metal zeolite mangostin, metal zeolite shikonin, metal zeolite anhydroalkanin, metal zeolite glycyrol, metal zeolite isoliquiritin, metal zeolite kuraridin, metal zeolite curcumin, metal zeolite hydroquinone, metal zeolite catechol, and combinations thereof.

In the present invention, said compounds of metal zeolite glycols and polyglycols are further selected from, but not limited to metal zeolite propylene glycol, metal zeolite butylene glycol, metal zeolite pentylene glycol, metal zeolite hexylene glycol, metal zeolite polyethylene glycol, metal zeolite polypropylene glycol, metal zeolite glycerin, metal zeolite diglycerin, metal zeolite polyglycerin, metal zeolite sorbitol, metal zeolite mannitol, metal zeolite sucrose esters, metal zeolite polysorbates, metal zeolite mono-, di- and tri-ethylene glycol monoalkyl ethers, metal zeolite methylpropanediol, metal zeolite ethylhexylglycerin, and combinations thereof.

In the present invention, said metal zeolite amino acids are further selected from, but not limited to metal zeolite glycine, metal zeolite alanine, metal zeolite beta-alanine, metal zeolite valine, metal zeolite leucine, metal zeolite isoleucine, metal zeolite phenylalanine, metal zeolite alpha-amino butyric acid, metal zeolite C-phenylglycine, metal zeolite C-hydroxyphenylglycine, metal zeolite proline, metal zeolite tryptophane, metal zeolite lysine, metal zeolite ornithine, metal zeolite arginine, metal zeolite histidine, metal zeolite citrulline, metal zeolite glutamic acid, metal zeolite aspartic acid, metal zeolite serine, metal zeolite threonine, metal zeolite hydroxyproline, metal zeolite tyrosine, metal zeolite dihydroxytyrosine, metal zeolite cysteine, metal zeolite cystine, metal zeolite methionine, metal zeolite homocysteine, metal zeolite lanthionine, or combinations thereof.

In the present invention, said metal zeolite peptides are further selected from, but not limited to metal zeolite dipeptides, metal zeolite tripeptides, metal zeolite tetrapeptides, metal zeolite pentapeptides, metal zeolite hexapeptides, metal zeolite heptapeptides, metal zeolite octapeptides, metal zeolite nonapeptides, and such, including metal zeolite insulin, metal zeolite bradykinin, metal zeolite glutathione, and metal zeolite carnosine as examples. Pickart et al. have disclosed a number of other such peptides (U.S. Pat. Nos. 5,858,993; 5,888,522; 5,698,184; 5,550,183; 5,554,375; 5,164,367; 4,665,054; 4,760,051; 4,810,693 and 4,877,770; U.S. patent application Ser. No. 20050276766), metal zeolites of which can be further utilized in the present invention.

In the present invention, said metal zeolite vitamins are further selected from, but not limited to metal zeolite Vitamin A, metal zeolite Retinol, metal zeolite Retinoic acid, metal zeolite Tretinoin, metal zeolite of members of Vitamins B group, metal zeolite Vitamin C, metal zeolite Vitamin D, metal zeolite Vitamin E, metal zeolite Vitamin K, metal zeolite Carotenes, metal zeolite Biotin, metal zeolite Folic Acid, and combinations thereof.

In the present invention, said metal zeolite hormones are further selected from, but not limited to metal zeolite progesterone, metal zeolite androsterone, metal zeolite dehydroepiandrosterone (DHEA), metal zeolite pregnenolone, metal zeolite androstenedione, metal zeolite melatonin, metal zeolite testosterone, and combinations thereof.

In the method of the present invention, a compound of metal zeolite is applied topically at a desired site in a sufficient quantity, and wherein said application can be done either by a manual or a mechanical methods or a combination thereof. Among mechanical methods, electrically driven instruments, such as rotating or vibrating disks, rotating or vibrating pads, and such, are included. Among manual methods, use of hands or fingers, spatulas, spoons, and such, are included.

However, it is to be noted that it is not the intention to include each and every possible specific example of various chemical groups or classes of compounds of metal zeolite compounds or methods of application mentioned herein.

The metal zeolite compounds of the present invention are made, among other processes, by first forming a metal zeolite complex by the reaction of a sodium or potassium zeolite, or a mixture of Na and K zeolite, with a metal donor compound; wherein said metal compound is a di- or a polyvalent metal. This is shown in [Equation 1].

Na/K Zeolite+Metal Donor Compound→Metal Zeolite Complex [Equation 1].

The metal donor compound is preferably selected from a metal salt or compound of a hydroxy, polyhydroxy, or amino acid; for example, various metal gluconates, metal salicylates, metal lactates, and metal amino acetates.

The metal zeolite complex [from Equation 1] is then reacted with a compound having an O-alkyl, O-cycloalkyl, O-aralkyl, O-aryl, O-heterocyclic, O-vinyl, O-vinyl alkyl, O-vinyl aryl, O-vinyl heterocyclic, O-keto alkyl, O-keto aryl, O-keto heterocyclic, O-keto peptide, O-keto alkyl amine, O-phosphate ester, O-carboxy alkyl, O-carboxy aryl, S-alkyl, S-cycloalkyl, S-aralkyl, S-aryl, S-heterocyclic, S-vinyl, S-vinyl alkyl, S-vinyl aryl, S-vinyl heterocyclic, S-keto alkyl, S-keto aryl, S-keto heterocyclic, or S-keto alkyl amine group to form zeolite metal compounds of the present invention, [Equation 2].

Alternatively, a metal salt or compound of a compound having an O-alkyl, O-cycloalkyl, O-aralkyl, O-aryl, O-heterocyclic, O-vinyl, O-vinyl alkyl, O-vinyl aryl, O-vinyl heterocyclic, O-keto alkyl, O-keto aryl, O-keto heterocyclic, O-keto peptide, O-keto alkyl amine, O-phosphate ester, O-carboxy alkyl, O-carboxy aryl, S-alkyl, S-cycloalkyl, S-aralkyl, S-aryl, S-heterocyclic, S-vinyl, S-vinyl alkyl, S-vinyl aryl, S-vinyl heterocyclic, S-keto alkyl, S-keto aryl, S-keto heterocyclic, or S-keto alkyl amine group can be used in Equation 1 to form the corresponding metal zeolite compound of the present invention.

Metal Zeolite Complex+A compound having an O-alkyl, O-cycloalkyl, O-aralkyl, O-aryl, O-heterocyclic, O-vinyl, O-vinyl alkyl, O-vinyl aryl, O-vinyl heterocyclic, O-keto alkyl, O-keto aryl, O-keto heterocyclic, O-keto peptide, O-keto alkyl amine, O-phosphate ester, O-carboxy alkyl, O-carboxy aryl, S-alkyl, S-cycloalkyl, S-aralkyl, S-aryl, S-heterocyclic, S-vinyl, S-vinyl alkyl, S-vinyl aryl, S-vinyl heterocyclic, S-keto alkyl, S-keto aryl, S-keto heterocyclic, or/and S-keto alkyl amine group→Zeolite Metal Compound [Equation 2].

Certain examples of these preparations (via Equations 1 and 2) are shown in Scheme 1:

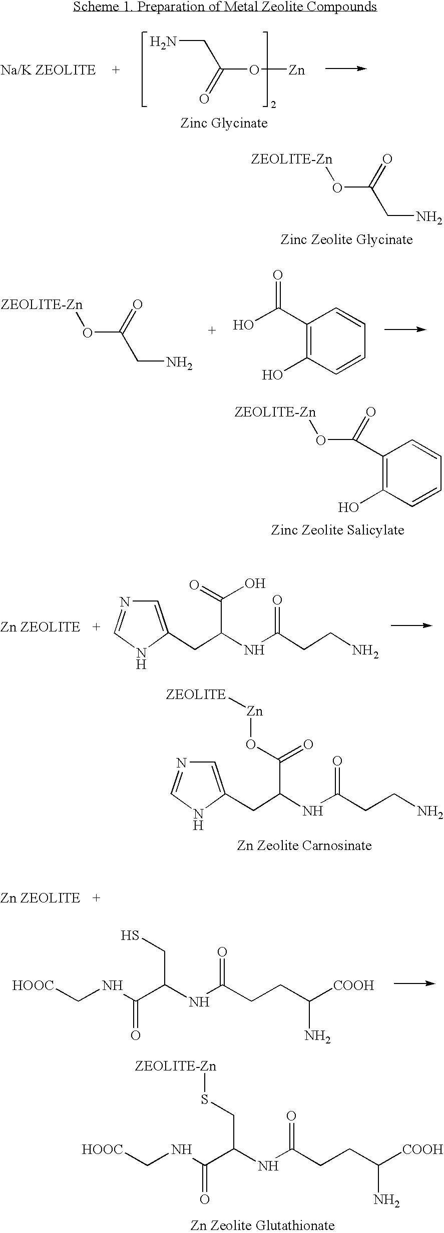

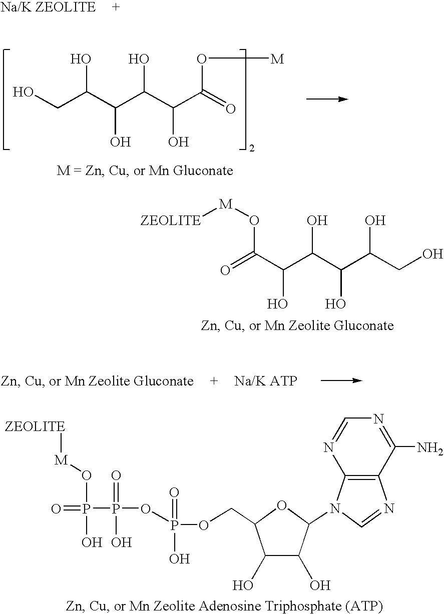

The inclusion of a hydroxylic solvent is required during the reactions of Equations 1 and 2, and Scheme 1. The said hydroxylic solvents include, but not limited to, various glycols, polyglycols, glycerin, diglycerin, polyglycerin, esters of hydroxy acid, various alcohols, water, pyrrolidone, and N-methylpyrrolidone. Diglycerin, polyethylene glycol, ethyl lactate, and triethyl citrate are most preferred hydroxylic solvents when anhydrous conditions are required, for example, in the preparation of anhydrous metal zeolite compounds with heat releasing property.

The presence of any strong chelating agent during the above chemical reactions can inhibit or retard these reactions.

The heating of these reaction mixtures at from 30 degrees Celsius to 95 degrees Celsius can cause an acceleration of reaction rates.

Relative to the type of Na/K zeolites that can be used in the present invention, the synthetic zeolites consisting of zeolite, type 3A; zeolite type 4A; zeolite, type 5A; zeolite, type 9A; zeolite, type 13×; zeolite, type CAX; zeolite, type LSX; zeolite, type HPZ, and other similar zeolites with controlled pore sizes of from 3 Angstroms to 30 Angstroms are preferred. Zeolite, type 4A and 9A are most preferred. For anhydrous metal zeolite compounds of the present invention, both the use of anhydrous Na/K zeolites and the absence of water during processing are preferred.

The three-dimensional chemical structure of certain metal zeolite enzyme inhibitors, for example, is yet unknown. This is due to the lack of knowledge of three-dimensional chemical structure of said enzymes themselves. This, however, should not make the importance of the present invention any less meaningful.

Relative to the chemical bonding of the metal, the zeolite, and the substituent to each other in a specific metal zeolite compound it is known that such bonding is via one or more —Si—O— moieties of zeolite. For example, Na/K zeolite, Na/K zeolite with partial replacement by Zn, and fully exchanged Zn zeolite can be represented by the following structural formulas, wherein only a few —Si—O— bonds on zeolite framework are shown for clarity purposes.

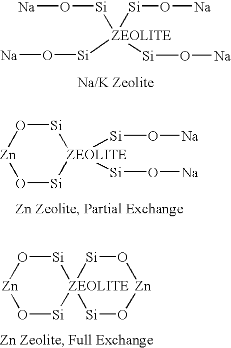

On the same note, the binding of an O-alkyl, O-cycloalkyl, O-aralkyl, O-aryl, O-heterocyclic, O-vinyl, O-vinyl alkyl, O-vinyl aryl, O-vinyl heterocyclic, O-keto alkyl, O-keto aryl, O-keto heterocyclic, O-keto peptide, O-keto alkyl amine, O-phosphate ester, O-carboxy alkyl, O-carboxy aryl, S-alkyl, S-cycloalkyl, S-aralkyl, S-aryl, S-heterocyclic, S-vinyl, S-vinyl alkyl, S-vinyl aryl, S-vinyl heterocyclic, S-keto alkyl, S-keto aryl, S-keto heterocyclic, or S-keto alkyl amine moiety on a metal zeolite compound of formula (I), for example, zinc zeolite salicylate, is via —Si—O—Zn— moiety of said zeolite, as illustrated below.

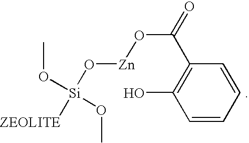

In the method comprising a compound, or a salt thereof; or a composition comprising a compound or a salt thereof; of the present invention, for the treatment of skin or hair condition by topical application; wherein,

(i) Said application having been done either by a manual or a mechanical method, or a combination thereof; and, wherein,

(ii) Said application is repeated as necessary to treat said condition.

In the method of the present invention said skin or hair condition is selected from the group consisting of alleviation of skin conditions such as skin rash including diaper rash, dry skin, scalp dandruff, broken or chafed skin, sunburn, skin damage from UV, skin irritation, acne including excess facial oil and facial pore size; darkened skin including age spots, dark circles around the eyes, and discoloration of skin from stretch marks; skin aging including wrinkles and fine lines; loss of collagen including thinning skin and loss of skin pliability; body odor, including oral cavity odor, arm-pit odor, and incontinence odor; cellular inflammation including intracellular and extra cellular inflammation; premature hair aging including premature hair loss hair graying; malfunction of tyrosinase group of enzymes, malfunction of matrix metalloprotease group of enzymes; and combinations thereof. The said method also provides topical delivery of certain metals, including trace metals, and certain zirconium aluminum amino acids that provide antiperspirant benefits.

In the method according to present invention, said skin or hair condition is darkened skin including age spots, dark circles around the eyes, and discoloration of skin from stretch marks.

In the method according to present invention, wherein said skin or hair condition is skin aging including wrinkles and fine lines.

In the method according to present invention, wherein said skin or hair condition is premature hair aging including premature hair loss and hair graying.

In the method according to present invention, wherein said skin condition is darkened skin including age spots, dark circles around the eyes, and discoloration of skin from stretch marks.

In the method according to present invention, the compounds of the present invention undergo a unique chemical reaction when Na or K ions that are released with the body's natural perspiration come in contact with said compounds, as illustrated in Scheme 2:

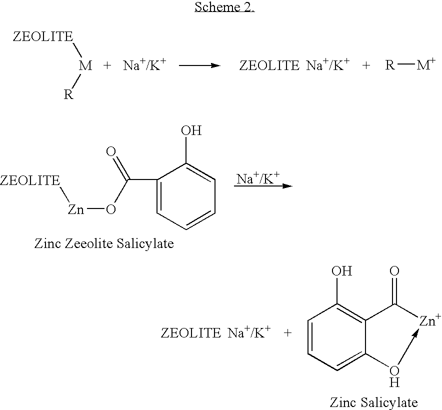

In the method according to present invention the compounds thus delivered to skin via Scheme 2 provide their own treatment benefits. The clinical testing of said compounds has not been performed, as ample prior art references are available relative to the treatment benefits of compounds thus delivered to skin or hair.

In the present invention a variety of delivery systems and carrier base forms can be utilized. Such forms include the group consisting of shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system or a carrier base of the present are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, skin penetration enhancing agent, impregnated or coated diaper, and similar delivery or packaging form.

In another preferred aspect, the delivery system of the present invention can be traditional water and oil emulsions, suspensions, colloids, microemulsions, clear solutions, suspensions of nanoparticles, emulsions of nanoparticles, or anhydrous compositions.

Additional ingredients or agents can also be included in the present invention, which can be selected from, but not limited to skin penetration enhancers, skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, solubilizers, anesthetics, colorants, perfumes, preservatives, seeds, broken seed nut shells, silica, clays, beads, luffa particles, polyethylene balls, mica, pH adjusters, processing aids, and combinations thereof.

In the method of the present invention a skin penetration enhancing agent can be included, which is selected from methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, triethyl citrate, trimethyl citrate, tributyl citrate, acetyl triethyl citrate, acetyl tributyl citrate, trihexyl citrate, butyl trihexyl citrate, stearyl citrate, diethyl tartrate, dimethyl tartrate, ethyl mandelate, ethyl salicylate, methyl salicylate, ethyl glycolate, and combinations thereof. Additional skin penetration agents suitable for use are included in a prior disclosure by the present inventor (U.S. patent application Ser. No. 20060110415), which is included in its entirety herein.

In another preferred aspect, in the present invention one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and phospholipids can be included.

Representative saccharides include nonionic or cationic saccharides such as agarose, amylopectins, amyloses, arabinans, arabinogalactans, arabinoxylans, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomainans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994), which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable carriers of this invention may include surface-active agents. Surface-active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metals and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable carriers of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecylbenzene sulfonate.

Amphoteric surface-active agents that may be used in the cosmetically acceptable carriers of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable carriers of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyldimonium chloride, Dihydrogenated tallow benzylmonium chloride, disoyadimonium chloride, Ditallowedimonium chloride, Hydroxycetyl hydroxyethyl dimonium chloride, Hydroxyethyl Behenamidopropyl dimonium chloride, Hydroxyethyl Cetyldimonium chloride, Hydroxyethyl tallowedimonium chloride, myristalkonium chloride, (PEG=Polyethylene glycol) PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryldimonium chloride, cetalkonium chloride, dicetyldimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable carriers of the present invention may include long chain fatty amines from about C.sub.10 to C.sub.22 and their derivatives. Specific examples include dipalmitylamine, lauramidopropyldimethylamine, and stearamidopropyl dimethylamine. The cosmetically acceptable carriers of this invention may also include fatty alcohols (typically monohydric alcohols), ethoxylated fatty alcohols, and di-tail phospholipids, which can be used to stabilize emulsion or dispersion forms of the cosmetically acceptable carriers. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof.

Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable carriers of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable carrier of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable carrier of this invention are typically used in an amount from about 0.1 to 50 percent by weight, preferably from about 0.5 to about 40 percent by weight, more preferably from about 1 to about 20 percent by weight.

The cosmetically acceptable carrier of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable carrier of this invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, Vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable carrier of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The carriers of this invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane end blocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable carriers of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane-200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable carriers of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in the carriers of the present invention. Preferred examples include dimethicone copolyols and 5225C Formulation Aids, available from Dow Corning, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Corning 8220, Dow Corning 939, Dow Corning 949, Dow Corning 2-8194, all available from Dow Corning, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable carriers of this invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the trade name Permethyl 101A, available from Presperse, South Plainfield, N.J., USA. Another example of a preferred volatile hydrocarbon is C.sub.12 to C.sub.14 isoparaffin, under the trade name Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable carriers of this invention may include cationic and ampholytic conditioning polymers. Examples of such include, but are not limited to those listed by the International Cosmetic Ingredient Dictionary published by the Cosmetic, Toiletry, and Fragrance Association (CTFA), 1101 17 Street, N.W., Suite 300, Washington, D.C. 20036. General examples include quaternary derivatives of cellulose ethers, and quaternary derivatives of guar. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable carrier of this invention at a concentration of from 0.1 to 10 weight percent, preferably from 0.2 to 6 weight percent and most preferably from 0.2 to 5 weight percent.

The cosmetically acceptable carrier of this invention may include one or more rheological modifiers. The rheological modifiers that can be used in this invention include high molecular weight crosslinked homopolymers of acrylic acid, and Acrylates/C10-30 Alkyl Acrylate Crosspolymer, such as the Carbopol and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropyltrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, poly(ethylene oxide) based thickeners, sodium carbomer, and mixtures thereof. When used, the rheology modifiers are preferably included in the cosmetically acceptable carrier of this invention at a concentration of from 0.01 to 12 weight percent, preferably from 0.05 to 10 weight percent and most preferably from 0.1 to 6 weight percent.

The cosmetically acceptable carrier of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 20 weight percent, preferably 0.5 to 10 weight percent and most preferably from 1.0 to 5.0 weight percent of the cosmetically acceptable carrier.

The cosmetically acceptable carrier of this invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl para-aminobenzoate (PABA), diethanolamine p-methoxy cinnamate, ethyl 4-bis(hydroxypropyl)aminobenzoate, 2-ethylhexy 1-2-cyano-3,3-diphenylacrylate, homomethyl salicylate, glyceryl aminobenzoate, dihydroxyacetone, octyl dimethyl PABA, 2-phenylbenzimidazole-5-sulfonic acid, triethanolamine salicylate, zinc oxide, zinc zeolite, titanium zeolite, and titanium oxide, and mixtures thereof. The amount of sunscreen used in the cosmetically acceptable carrier of this invention will vary depending on the specific UV absorption wavelength(s) of the specific sunscreen active(s) used and can be from 0.1 to 10 percent by weight, from 2 to 8 percent by weight.

The cosmetically acceptable carrier of this invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis(hydroxymethyl)-5,5-dimethyl-2,3-imidazolidinedione (e.g., DMDM Hydantoin, known as GLYDANT, Lonza, Fairlawn, N.J., USA), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable carrier of this invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The cosmetically acceptable carrier of this invention can be presented in various forms. Examples of such forms include, but are not limited a solution, liquid, cream, emulsion, dispersion, gel, thickening lotion.

The cosmetically acceptable carrier of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerin, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture from 0.1 to 70 percent by weight, relative to the weight of the total composition.

The cosmetically acceptable carrier of this invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable carrier of this invention can contain electrolytes, such as aluminum chlorohydrate. Among most notable are certain very popular antiperspirant compounds of aluminum zirconium amino acids, which, for example U.S. Pat. Nos. 5,156,834; 4,675,177; and 4,724,139, are represented by generic formula: Al.sub.nZr(OH).sub.(3n+4-x)Cl.sub.x(AA).sub.y; wherein n is from 3 to 10, preferably 6 to 10; x is from 3 to 9, calculated from M/Cl ratio, of at least 1:1; AA is a buffer, which includes an amino acid, y is from 0.055 to 0.22 calculated from y/M molar ratio, which is from 0.05 to 0.2, preferably from 0.05 to 0.15. Aluminum salts of this type include aluminum chloride and the aluminum hydroxyhalides having the general formula Al.sub.2 (OH).sub.xQ.sub.y.sup..XH.sub.2O where Q is chlorine, bromine or iodine; where x is 2 to 5 and x+y=6 and x and y do not need to be integers; and where X is about 1 to 6. Aluminum salts of this type can be prepared in the manner described more fully in Gilman, U.S. Pat. No. 3,887,692, issued Jun. 3, 1975, and U.S. Pat. No. 3,904,741, Sep. 9, 1975 to Jones and Rubino incorporated herein by reference. The zirconium compounds, which are useful in the present invention, include both the zirconium oxy salts and zirconium hydroxy salts, also referred to as the zirconyl salts and zirconyl hydroxy salts. Although only zirconium compounds are exemplified in this specification, it will be understood that other Group IV B metals, including hafnium could be used in the present invention. As with the basic aluminum compounds, it will be understood that the above formula is greatly simplified and is intended to represent and include compounds having coordinated and/or bound water in various quantities, as well as polymers, mixtures and complexes of the above. As will be seen from the above formula, the zirconium hydroxy salts actually represent a range of compounds having various amounts of the hydroxy group, varying from about 1.1 to only slightly greater than 0 groups per molecule. Several types of antiperspirant complexes utilizing the above antiperspirant salts are known in the art. For example Luedders et al; U.S. Pat. No. 3,792,068, issued Feb. 12, 1974 discloses complexes of aluminum, zirconium and amino acids such as glycine. Complexes such as those disclosed in this Luedders et al '068 patent and other similar complexes are commonly known as ZAG. ZAG complexes are chemically analyzable for the presence of aluminum, zirconium and chlorine. ZAG complexes useful herein are identified by the specification of both the molar ratio of aluminum to zirconium (hereinafter “Al:Zr” ratio) and the molar ratio of total metal to chlorine (hereinafter “Metal:Cl” ratio). ZAG complexes useful herein have an Al:Zr ratio of from about 1.67 to 12.5 and a Metal:Cl ratio of from about 0.73 to 1.93.

A preferred aluminum compound for preparation of such ZAG type complexes is aluminum chlorhydroxide of the empirical formula Al.sub.2 (OH).sub.5Cl.sup..2H.sub.2O. Preferred zirconium compounds for preparation of such ZAG-type complexes are zirconyl hydroxychloride having the empirical formula ZrO(OH)Cl.sup..3H.sub.2O and the zirconyl hydroxyhalides of the empirical formula ZrO(OH).sub.2-aCl.sub.2.sup..nH.sub.2O wherein a is from 1.5 to 1.87 and n is from about 1 to 7. The preferred amino acid for preparing such ZAG-type complexes is glycine of the formula CH.sub.2(NH.sub.2)COOH. Salts of such amino acids can also be employed in such antiperspirant complexes. See U.S. Pat. No. 4,017,599 to A. M. Rubino issued Apr. 12, 1977 specifically incorporated herein by reference.

The preferred zirconium aluminum zeolite amino acid of the present invention are represented by general formula:

ZEOLITE-[Al]n.Zr(OH)₃n+4−x.Clx[AA]y,

Wherein,

n=3 to 10; x=3 to 9; AA=amino acid; and y=0.05 to 3.

The specific zirconium aluminum amino acid moieties are selected from Aluminum Zirconium Tetrachlorohydrex Glycine and Aluminum Zirconium Trichlorohydrex Glycine, available from Reheis Chemical Company, 235 Snyder Ave, Berkeley Heights, N.J. 07922, USA.

A wide variety of other types of antiperspirant complexes are also known in the art. For example, Siegal; U.S. Pat. No. 3,903,258, issued Sep. 2, 1975 discloses a zirconium aluminum complex prepared by reacting zirconyl chloride with aluminum hydroxide and aluminum chlorhydroxide. Rubino; U.S. Pat. No. 3,979,510, issued Sep. 7, 1976 discloses an antiperspirant complex formed from certain aluminum compounds, certain zirconium compounds and certain complex aluminum buffers. Rubino; U.S. Pat. No. 3,981,896, issued Sep. 21, 1976 discloses an antiperspirant complex prepared from an aluminum polyol compound, a zirconium compound and an organic buffer. Mecca; U.S. Pat. No. 3,970,748, issued Jul. 20, 1976 discloses an aluminum chlorhydroxy glycinate complex of the approximate general formula [Al.sub.2 (OHhd)Cl][H.sub.2 CNH.sub.2COOH]. All of these patents are incorporated herein by reference. Of all the above types of antiperspirant actives, preferred compounds include the 5/6 basic aluminum salts of the empirical formula Al.sub.2 (OH).sub.5Cl.sup.. 2H.sub.2O; mixtures of AlCl.sub.3.sup..6H.sub.2O and Al.sub.2(OH).sub.5 Cl.sup..2H.sub.2O with aluminum chloride to aluminum hydroxychloride weight ratios of up to about 0.5; ZAG type complexes wherein the zirconium salt is ZrO(OH)Cl.sup..3H.sub.2O; the aluminum salt is Al.sub.2(OH).sub.5Cl.sup.. 2H.sub.2O or the aforementioned mixtures of AlCl.sub.3.sup..6H.sub.2O and Al.sub.2(OH).sub.5Cl.sup..2H.sub.2O wherein the total metal to chloride molar ratio in the complex is less than about 1.25 and the Al:Zr molar ratio is about 3.3; and the amino acid is glycine and ZAG-type complexes wherein the zirconium salt is ZrO(OH).sub.2-aCl.sub.a.sup..nH.sub.2O with a ranging from about 1.5 to 1.87 and n ranging from about 1 to 7; the aluminum salt is Al.sub.2(OH).sub. 5Cl.sup..2H.sub.2O; and the amino acid is glycine.

The carriers of this invention for treating skin include leave-on or rinse-off forms such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, said carriers may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The carriers of this invention may be prepared as oil-in-water, water-in-oil emulsions, triple emulsions, or dispersions. Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

The carriers of this invention may be a shampoo, which may contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The carriers of this invention may be a shampoo for washing hair, which can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

The carriers of this invention may consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the carriers are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acrylnitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The carriers of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonite, silica, talc, zinc oxide or mixtures of these ingredients.

The carriers of this invention, in the form of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The carriers of this invention as solution or emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The carriers of this invention as suspensions may comprise liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminum metahydroxide, bentonite, agar and tragacanth or mixtures of these ingredients.

The carriers of this invention may contain additional antioxidants, which are selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The carriers of this invention may contain the blood micro-circulation improvement ingredients which are selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The carriers of this invention may include the anti-inflammatory agents, which are selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

### EXAMPLES

The following examples illustrate presently preferred practice thereof. As illustrations they are not intended to limit the scope of the invention. All quantities are in weight percent. C is temperature in degree Celsius.

### Example 1

### Preparation of Zinc Zeolite Salicylate (Scheme 1)

Ingredients (1) Zeolite, 4A, anhydrous 10.0 (2) Zinc Salicylate 2.0 (3) Polyethylene glycol 88.0. Procedure. Mix (1) to (3) and heat at 75 to 85 C. Cool to room temperature. Filter. Zinc Zeolite Salicylate is obtained as an off-white powder.

### Example 2

### Preparation of Zinc Zeolite Glycinate (Scheme 1)

Ingredients (1) Zeolite, 4A, anhydrous 10.0 (2) Zinc Glycinate 2.0 (3) Polyethylene glycol 88.0. Procedure. Mix (1) to (3) and heat at 75 to 85 C. Cool to room temperature. Filter. Zinc Zeolite Glycinate is obtained as an off-white powder.

### Example 3

### Alternate Preparation of Zinc Zeolite Salicylate (Scheme 1)

Ingredients (1) Zinc Zeolite Glycinate 10.0 (2) Salicylic Acid 5.0 (3) Polyethylene glycol 85.0. Procedure. Mix (1) to (3) and heat at 75 to 85 C. Cool to room temperature. Filter. A mixture of Zinc Zeolite Salicylate and glycine is obtained as an off-white powder.

### Example 4

### The Method of Topical Treatment with Zinc Zeolite Salicylate for Acne

The following steps are performed for this method of topical treatment. (1) Zinc zeolite salicylate 25.0 and a carrier lotion 75.0 are mixed together. (2) The composition is applied topically in the amount and frequency necessary to achieve desired treatment. (3) After the application, the composition is left on skin for several hours before washing.

### Example 5

### The Method of Topical Treatment with Zinc Zeolite Tyrosinase Inhibitor Compounds for Treating a Combination of Skin Wrinkles, Darkened Skin, and Skin Damage from UV

The following steps are performed for this method of topical treatment. (1) A combination of zinc zeolite resacetophenone 2.0, zinc zeolite lactate 5.0, zinc zeolite hydroquinone 2.0, and carrier lotion 91.0, are mixed together. (2) The mixture is applied manually in the amount and frequency necessary to achieve desired treatment. (3) After the application, the composition is left on skin for several hours, and then the application is repeated as necessary.

### Example 6

### The Method of Topical Treatment with Zinc Zeolite Matrix Metalloprotease Inhibitor, Zinc Zeolite Lignan Compounds and a Penetration Enhancing Agent for Treatment of Diaper Rash, Chafed Skin, and Dry Skin

The following steps are performed for this method of topical treatment. (1) The zinc zeolite phloridzin 2.0, zinc zeolite salicylate 5.0, zinc zeolite sesamin 2.0, triethyl citrate 5.0 (penetration enhancing agent) and carrier lotion 86.0, are mixed together. (2) The mixture is applied on a diaper, which is then placed on afflicted area of skin. (3) After the placement of said diaper on skin, it is left on skin for several hours before removing or washing, or on as needed basis to replace any soiled diaper.

### Example 7

### The Method of Topical Treatment with Zinc Zeolite Glycol for Body Odor Treatment

The following steps are performed for this method of topical treatment. (i) A solid stick carrier base is first prepared as follows. Ingredients (1) Sodium Stearate 8.0 (2) Propylene Glycol 7.5 (3) PEG-4 7.5 (4) Cyclomethicone 40.0 (5) Isostearyl Alcohol 19.5 (6) PPG-10 Cetyl Ether 10.0 (7) Water 7.5. Procedure. Mix (1) to (3) and heat at 70 to 80 C. Cool to 60 to 65 C and add all other ingredients. Zinc zeolite ethylhexylglycerin is then made by mixing (1) zinc zeolite 30.0 (2) Ethylhexylglycerin 15.0 and (3) Methylpropanediol 55.0 at 35 to 40 C. The above carrier base 75.0 and Zinc zeolite ethylhexylglycerin compound 25.0 (from step (ii) above) are mixed at 60 to 65 C. This mixture is poured into a plastic deodorant stick packaging and cooled to room temperature until it turns into a solid stick. The solid stick thus obtained is applied manually in the amount and frequency necessary to achieve the desired body odor treatment.

### Example 8

### Preparation of Zinc Zeolite Peroxide from Na/K Zeolite

Ingredients. (1) Zeolite, Type 4A, hydrated 20.0 (2) Zinc Peroxide 1.0 (3) Water 79.0. Procedure. Mix (2) and (3) to a clear solution. Add (1) and mix. The mixture contains 10.0 Mmol of Zinc Zeolite Peroxide.

### Example 9

### Preparation of Zinc Peroxide—Zeolite Complex from Zinc Zeolite

Ingredients. (1) Zinc Zeolite 10.0 (2) Sodium Peroxide 3.0 (3) Water 87. Procedure. Mix (2) and (3) to a clear solution. Add (1) and mix and heat at 30 to 40 C. The mixture contains about 30 Mmol of Zinc Zeolite Peroxide.

### Example 10

### Preparation of Zinc Zeolite Benzoyl Peroxide

Ingredients. (1) Zinc Zeolite, Type 4A, anhydrous 40.0 (2) Benzoyl Peroxide 2.4 (3) PEG-6 57.6. Procedure. Mix (2) and (3) to a clear solution. Add (1) and mix and heat at 30 to 40 C. The mixture contains 10 Mmol of Zinc zeolite Benzoyl Peroxide.

### Example 11

### Skin Whitening Serum

Ingredients. (1) Ethyl Lactate 20.0 (2) Polyalkyleneoxy Polyamide 0.5 (3) Zinc Zeolite Peroxide 9.0 (4) PEG-6 70.0 (5) Preservatives 0.5. Procedure. Make serum base by mixing (1), (2) and (4) at 60 to 70 C. Cool to 30 to 40 C and add (3) to main batch with mixing.

### Example 12

### Anti-Acne and Facial Oil Control Cream

Ingredients. (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Ethyl Lactate 5.0 (6) Zinc Zeolite Peroxide 4.0 (7) Preservatives 0.5. Procedure. Mix 1 to 5 and heat to 75-80° C. Adjust pH to 4.0 4.5. Cool to 35-40 C with mixing. Add 6 to 7 with mixing. An off-white cream is obtained.

### Example 13

### Skin Decolorizing and Age Spots Cream

Ingredients. (1) Water 53.9 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Zn Zeolite Benzoyl Peroxide 8.1 (16) Artemisinin 0.5 (17) Carnosine 0.1 (18) Cyclomethicone, Dimethicone Crosspolymer 2.0 (19) Polysorbate-20 2.0 (20) Ethyl Lactate 12.0. Procedure. Mix (1) to (13) and heat at 70 to 80 C till homogenous. Cool to 40 to 50 C. Premix and add all other ingredients to main batch and mix. Cool to room temperature. An off-white cream is obtained.

### Example 14

### Skin Whitening Cream

Ingredients. (1) Water 53.8 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (6) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Zinc Zeolite Peroxide 11.5 (15) Ethyl Lactate 15.0 (16) Cyclomethicone, Dimethicone Crosspolymer 2.0 (17) Polysorbate-20 2.0 (18) Polyacrylamide 2.0. Procedure. Mix (1) to (13) and heat at 70 to 80 C till homogenous. Cool to 40 to 50 C. Premix (14) to (17) and add to main batch with mixing. Cool to room temperature and add (18) and mix. An off-white cream is obtained.

### Example 15

### Skin Brightening Cleanser

Ingredients. (1) PEG-6 47.23 (2) Hydroxypropyl Guar 0.4 (3) Sodium Cocoyl Isethionate 20.0 (4) Sodium Lauryl Sulfoacetate 5.0 (5) Boswellia Serrata 0.05 (6) L-Glutathione 0.01 (7) Resveratrol 0.01 (8) Artemisinin 0.1 (9) 2,6-Dihydroxy Acetophenone 1.0 (10) Zinc Zeolite Urea Peroxide 10.0 (11) Phenoxyethanol 0.7 (12) Ethylhexylglycerin 0.3 (13) Fragrance 0.2 (14) Ethylhexyl Lactate 15.0. Procedure. Mix (1) and (2) to a clear thin gel. Add (3) and (4) and mix. Premix (5) to (14). Add to main batch and mix. A white cream-like cleanser is obtained.

### Example 16

### Facial Glow Fade Cream

Ingredients. (1) Water 72.45 (2) Dicetyl phosphate and Ceteth-10 phosphate 5.0 (3) Glyceryl Stearate and PEG-100 stearate 4.0 (4) Diglycerol 2.0 (5) Shea butter 2.0 (6) Calcium Zeolite Peroxide 3.0 (7) Copper glycinate 2.2 (8) Capuacu butter 1.0 (9) Sodium hydroxide 0.25 (10) Boswellia serrata extract 0.5 (11) Tetrahydrocurcumin 0.2 (12) Paeonol 0.2 (13) Coleus Forskohlii Root extract 0.1 (14) Polysorbate-20 4.0 (15) Carnosine 0.1 (16) Preservative 1.0 (17) Polyacrylamide and C13-14 Isoparaffin and Laureth-7 2.0. Procedure. Make Premix A by mixing (1) to (5) at 80 to 90 C. Cool to 40 to 50 C and add all other ingredients and continue mixing until homogenous. Cool to room temperature.

### Example 17

### A Method of Topical Decolorizing Treatment

The following steps are performed for this method of topical treatment. (1) The zinc zeolite peroxide 5.0 percent (Zeolite, pore size 9 Angstroms), water 75.0, and glycerin 20.0, are mixed together. (2) The mixture is applied topically in the amount necessary to achieve desired skin decolorization.

### Example 18

### A Topical Method for Skin Whitening Treatment

The following steps are performed. A combination of (1) PEG-6 50.0 (2) Vitamin A Palmitate 0.1 (3) Vitamin E Acetate 0.1 (4) Actiplex Botanicals 0.1 (5) Phenoxyethanol 0.5 (6) Liquapar 0.2 (7) Niacinamide 0.5, and (8) Hydroxypropyl cellulose 0.5, is mixed at 40 to 50 C for 6 hours, then (9) Calcium Zeolite percarbonate (zeolite from Atofina Nk30-pore size 13.0 Angstroms) 48.0 is added and mixing continued for an additional 2 hours. This is cooled to room temperature and applied topically at the site where skin decolorization is desired. The application is repeated as necessary to complete the said treatment.

### Example 19

### A Method for Skin Whitening Treatment

The following steps are performed. (A) The following are mixed: (1) PEG-6 90.0 (2) Dimethicone 2.0 (3) Vitamin A Palmitate 0.1 (4) Vitamin E Acetate 0.2 (5) Zinc zeolite Resacetophenone 5.0 (6) Phenoxyethanol 0.5 (7) Parabens 0.2 (8) Copper Zeolite ATP 1.0 (9) Zinc zeolite Glutathionate 0.5 (10) Licorice Root Extract 0.5, at 40 to 50 C for 6 hours, then cooled to room temperature. (B) The composition is applied topically where skin depigmentation is desired. (C) The application is repeated as necessary to complete the desired treatment.

### Example 20

### Preparation of Oxo Vanadium Zeolite Pyridoxal-5-Phosphate

Ingredients. (1) Na/K Zeolite, Type 4A 20.0 (2) vanadium (IV) chloride 2.0 (3) Polyethylene glycol methyl ether 74.0 (4) Sodium pyridoxal-5-phosphate 2.0 (5) Water 2.0. Procedure. Mix (1) to (3) with heating at 40 to 50 C. Add (4) and mix with heating at 40 to 50 C. Cool to room temperature. Add water with mixing. A suspension of Oxo Vanadium Zeolite Pyridoxal-5-phosphate is thus obtained.

### Example 21

### Preparation of Vanadium Zeolite Pyridoxal-5-Phosphate

Ingredients. (1) Na/K Zeolite, Type 4A 20.0 (2) vanadium (II) chloride 4.0 (3)

Polyethylene glycol methyl ether 74.0 (4) Sodium pyridoxal-5-phosphate 2.0. Procedure. Mix (2) and (3) to a clear solution. Add (1) and mix with heating at 40 to 50 C. Add Sodium pyridoxal-5-phosphate and continue mixing. Cool to room temperature. A suspension of Vanadium Zeolite Pyridoxal-5-phosphate is thus obtained.

### Example 22

### Preparation of Chromium Zeolite Benfotiamine

Ingredients. (1) Na/K Zeolite, Type 4A 20.0 (2) chromium picolinate 4.0 (3) Polyethylene glycol 74.0 (4) Benfotiamine 2.0. Procedure. Mix (1) to (3) with heating at 30 to 40 C. Add (4) and mix with heating at 40 to 50 C. Cool to room temperature. A suspension of Chromium Zeolite Benfotiamine is thus obtained.

### Example 23

### Preparation of Chromium Zeolite Picolinate

Ingredients. (1) Na/K Zeolite, Type 4A 20.0 (2) chromium picolinate 4.0 (3) Polyethylene glycol 76.0. Procedure. Mix (1) to (3) with heating at 30 to 40 C. Cool to room temperature. A suspension of Chromium Zeolite Picolinate is thus obtained.

### Example 24

### Preparation of Zinc Zeolite Triclosan

Ingredients. (1) Zn Zeolite 20.0 (2) Triclosan 4.0 (3) Polyethylene glycol 76.0. Procedure. Mix (1) to (3) with heating at 30 to 40 C. Cool to room temperature. A suspension of Zinc Zeolite Triclosan (formula XXVIII, M=Zn) is thus obtained. This is filtered and dried to give zinc zeolite triclosan as an off-white powder:

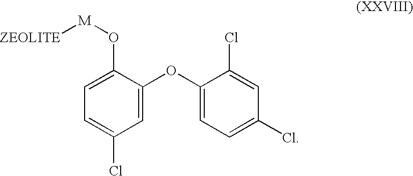

### Example 25

### Deodorant Aerosol Composition for Human or Animal Use

Ingredients (1) SD Alcohol 42.0 (2) Aminomethylpropanol 0.38 (3) Acrylates/Octylacrylmide Copolymer 2.0 (4) Zinc Zeolite Triclosan 10.0 (5) Isobutene (propellant) 45.62. Mix (1) and (2). Slowly add (3) with mixing. Add (4) and mix. Fill cans with the resulting composition and (5).

### Example 26

### Deodorant Roll-On or Spray Composition for Human or Animal Use

Ingredients (1) Water 43.65 (2) Methyl Parabens 0.15 (3) Diazolidinyl Urea 0.2 (4) EDTA 0.05 (5) Acrylates/C10-30 Alkyl Acrylate Crosspolymer 0.3 (6) Triethanolamine 0.3 (7) Ethylhexyl Hydroxystearate 4.0 (8) Propyl Parabens 0.1 (9) Capric/Caprylic Triglyceride 3.25 (10) Witch Hazel Distillate 28.0 (11) Zinc Zeolite Triclosan 10.0 (12) Zinc zeolite ethylhexylglycerin 10.0. Procedure. Mix (1) to (6) to a clear solution. Add (7) to (9) and mix at high speed at 40 to 50 C. Cool to 30-35 C and add all other ingredients and mix. Cool to room temperature. Fill in roll-on or spray delivery system packaging.

### Example 27

### Mouth Deodorizing and Triple Whitening Natural Toothpaste Composition for Human or Animal Use

Ingredients (1) Sorbitol 25.0 (2) Sodium Saccharin 0.27 (3) Trisodium Phosphate 1.0 (4) Potassium Cocoate 10.0 (5) Glycerin 10.0 (6) Cellulose Gum 0.3 (7) Water 24.28 (8) Titanium Dioxide 0.95 (9) Silica (fine abrasive) 12.0 (10) Silica (coarse abrasive) 4.0 (11) Peppermint Oil 0.5 (12) Spearmint 0.25 (13) Menthol 0.25 (14) zinc zeolite Urea Peroxide 0.5 (15) zinc zeolite triclosan 0.5 (16) Benzalkonium Chloride 0.2 (17) Zinc Zeolite (10.0). Procedure. Mix (7) and (8) first, then add all other ingredients and mix in a high-speed homogenizer. Fill in plastic tubes.

### Example 28

### Body Deodorant Sachet or Stick Composition

Ingredients (1) Zinc Zeolite ethylhexylglycerin 10.0 (2) Zinc zeolite triclosan 10.0 (3) Alkyl Benzoate 49.9 (4) Ethylenediamine/Hydrogenated Dimer Dilinoleate Copolymer Bis-Di-C14-C18 Alkyl Amide 10.0 (5) Exotic Butter Blend (Mango butter, Coco butter, Shea butter) 0.1 (6) Fragrance 20.0. Procedure. Mix (2) to (4) and heat at 70 to 80 C to a clear solution. Cool to 30 to 40 C. Add all other ingredients and mix. Fill in sachet, tube, tub, or stick packaging and cool to room temperature.

### Example 29

### Deodorant Talcum Body Powder

Ingredients (1) Corn Starch 66.0 (2) Talc 14.0 (3) PEG-62.0 (4) Zinc Zeolite triclosan 15.0 (5) Tetrahydrocurcumin 0.5 (6) Vitamin K-10.5 (7) Dimethicone 2.0. Procedure. Mix (1) and (2). Premix (3) to (6) and add to main batch and mix. A powder composition is obtained.

### Example 30

### Preparation of Zn, Cu, or Mn Zeolite Adenosine Triphosphate, ATP (Scheme 1)

Ingredients (1) Zeolite, 4A, anhydrous 10.0 (2) Zn, Mn, or Cu ATP 2.0 (3) Polyethylene glycol 88.0. Procedure. Mix (1) to (3) and heat at 75 to 85 C. Cool to room temperature. Filter. Zn, Cu, or Mn Zeolite ATP is obtained as an off-white powder.

### Example 31

### Preparation of Zinc, Cu, or Mn Zeolite Gluconate (Scheme 1)

Ingredients (1) Na/K Zeolite 10.0 (2) Zn, Cu, or Mn Gluconate 5.0 (3) Polyethylene glycol 85.0. Procedure. Mix (1) to (3) and heat at 75 to 85 C. Cool to room temperature. Filter. Zn, Cu, or Mn Zeolite Gluconate is obtained as an off-white powder.

### Example 32

### Alternate Preparation of Zn, Cu, or Mn Zeolite Adenosine Triphosphate, ATP (Scheme 1)

Ingredients (1) Zn, Cu, or Mn Zeolite Gluconate 10.0 (2) Na/K ATP 5.0 (3) Polyethylene glycol 85.0. Procedure. Mix (1) to (3) and heat at 75 to 85 C. Cool to room temperature. Filter. Zn, Cu, or Mn Zeolite ATP is obtained as an off-white powder.

### Example 33

### An Anhydrous Face Mask Controlled-Release Antiaging Composition with Heat-Releasing Effect

Ingredients. (1) Magnesium Sulfate (Anhydrous) 30.0 (2) Glycerin 49.0 (3) A 1:1:1 mixture of Zn, Cu, and Mn Zeolite Gluconate 20.0 (4) Antiaging Composition 1.0 (The antiaging composition is an equal weight mixture of Tetrahydrocurcumin, Niacinamide Lactate, Copper ATP complex, Glutathione, and Carnosine). Procedure: All ingredients are mixed in a dry atmosphere. A white paste is obtained. The face is rinsed with water first, and then the mask composition is applied as a film. The heat is felt immediately.

### Example 34

### Self-Heating Body, Hair and Facial Wash Product with Topical-Release Antiaging Ingredients and 1:1:1 Mixture of Zn, Cu, and Mn Zeolite Gluconate

Ingredients. (1) PEG-6 33.5 (2) Vitamin A Palmitate 0.1 (3) Vitamin E Acetate 0.1 (4) Actiplex Botanicals 0.1 (5) Phenoxyethanol 0.5 (6) Liquapar 0.2 (7) Niacinamide 0.5 (8) Zeolite (Atofina Nk30np) 32.0 (9) Sodium Lauryl Sulfoacetate 8.5 (10) Sodium Cocoyl Isethionate 14.0 (11) Citric Acid 4.0 (12) 1:1:1 mixture of Zn, Cu, and Mn Zeolite Gluconate 5.0 (13) Fragrance 0.5. Procedure: Mix all ingredients in a homogenizer mill. A paste is obtained.

### Example 35

### Self-Heating Body Butter with Skin Whitening Ingredients

Ingredients. (1) Castor Oil 20.8 (2) Mango Butter 2.0 (3) Cocoa Butter 4.0 (4) Beeswax 3.5 (5) Stimu-ex 0.2 (6) Avocado Butter 1.0 (7) Shea Butter 4.0 (8) Sweet Almond Oil 2.0 (9) Grapeseed Oil 2.0 (10) Dimethicone 5.0 (11) Hydrogenated Soybean Oil 6.0 (12) Sesame Oil 0.9 (13) Tinoguard TT 0.2 (14) Phenoxyethanol 0.5 (15) Propyl Paraben 0.2 (16) Aloe Vera (In Oil) 4.0 (17) Vitamin E Acetate 0.1 (18) Vitamin A Palmitate 0.1 (19) Zeolite (Atofina Nk30np) 30.0 (20) Lactic Acid 5.0 (21) Fragrance 3.5 (22) 1:1:1 mixture of Zn, Cu, and Mn Zeolite Gluconate 5.0. Procedure: Mix all ingredients and heat at 60 to 70 C. Cool to room temperature. A butter-like material is obtained.

### Example 36

### Self-Warming Anti-Acne Facial Wash

Ingredients. (1) Glycerin 22.288 (2) Methylpropanepdiol 15.0 (3) Vitamin A Palmitate 0.001 (4) Vitamin E Acetate 0.001 (5) Actiplex 0.01 (6) Phenoxyethanol 0.5 (7) Liquapar 0.2 (8) Zinc Zeolite Niacinate 0.5 (9) Zeolite Atofina Nk30np) 37.0 (10) Citric Acid 3.0 (11) Sodium Lauryl Sulfoacetate 7.0 (12) Veegum 1.0 (13) Sodium Cocoyl Isethionate 12.0 (14) Fragrance 1.5. Procedure: Mix all ingredients and heat at 50 to 60 C. Cool to room temperature. A paste-like product is obtained.

### Example 37

### Self-Warming Facial Anhydrous Mud Mask Product with Anti-Wrinkle Ingredients

Ingredients. (1) PEG-6 45.0 (2) Dimethicone 2.0 (3) Vitamin A Palmitate 0.001 (4) Vitamin E Acetate 0.001 (5) Actiplex 0.01 (6) Phenoxyethanol 0.5 (7) Liquapar 0.2 (8) Zeolite (Atofina Nk30np) 36.0 (9) Huber 90 White Clay 14.0 (10) Veegum 2.0 (11) Copper Zeolite ATP 0.1 (12) Manganese Zeolite Glutathionate 0.1 (13) Licorice Root Extract 0.5%. Procedure: Mix all ingredients at 50 to 60 C. Cool to room temperature. A paste-like material is obtained.

### Example 38

### Self-Heating Facial Clay Composition with Anti-Wrinkle and Anti-Oxidant Ingredients

Ingredients. (1) Glycerin 18.748 (2) Methylpropanediol 22.0 (3) Dimethicone 2.0 (4) Vitamin A Palmitate 0.001 (5) Vitamin E Acetate 0.001 (6) Dehydrated Aloe 0.01 (7) Cactus Extract 0.01 (8) Orange Extract 0.01 (9) Yucca Extract 0.01 (10) Prickly Pear Fruit Extract 0.01 (11) Fragrance 0.5 (12) Phenoxyethanol 0.5 (13) Liquapar 0.2 (14) Zeolite (Thermilux) 36.0 (15) Zinc Zeolite Lactate 0.5 (16) Mn Zeolite Citrate 2.5 (17) Mo Zeolite Glycolate 0.5 (18) Cu Zeolite Malate 0.5 (19) Huber 90 White Clay 14.0 (20) Veegum 2.0 Procedure: Mix all ingredients at 40 to 45 C. Cool to room temperature. A paste-like composition is obtained.

### Example 39

### Self-Warming Shampoo

Ingredients. (1) Glycerin 48.0 (2) Vitamin A Palmitate 0.1 (3) Zn Zeolite Vitamin E 0.1 (4) Actiplex 2794 (Plant Extracts) 0.1 (5) Phenoxyethanol 0.5 (6) Liquapar 0.2 (7) Mn Zeolite Niacinate 0.5 (8) Zeolite (WR Grace Silosiv) 30.0 (9) Sodium Lauryl Sulfoacetate 10.0 (10) Sodium Cocoyl Isethionate 10.0 (11) Fragrance 0.5. Procedure: Mix all ingredients in a homogenizer. A paste-like product is obtained.

### Example 40

### Preparation of Zirconium Aluminum Zeolite Trichlorohydrex Glycinate

Ingredients (1) Na/K Zeolite 10.0 (2) Aluminum-Zirconium Tetrachlorohydrex Glycine 5.0 (3) Polyethylene glycol 85.0. Procedure. Mix (1) to (3) and heat at 75 to 85 C. Cool to room temperature. Filter. Zirconium Aluminum Zeolite Trichlorohydrex Glycinate is obtained as an off-white powder.

### Example 41

### Deodorant Roll-On or Spray Composition with Zirconium Aluminum Zeolite Trichlorohydrex Glycinate

Ingredients (1) Water 43.65 (2) Methyl Parabens 0.15 (3) Diazolidinyl Urea 0.2 (4) EDTA 0.05 (5) Acrylates/C10-30 Alkyl Acrylate Crosspolymer 0.3 (6) Triethanolamine 0.3 (7) Ethylhexyl Hydroxystearate 4.0 (8) Propyl Parabens 0.1 (9) Capric/Caprylic Triglyceride 3.25 (10) Witch Hazel Distillate 28.0 (11) Zinc Zeolite Triclosan 10.0 (12) Zirconium Aluminum Zeolite Trichlorohydrex Glycinate 10.0. Procedure. Mix (1) to (6) to a clear solution. Add (7) to (9) and mix at high speed at 40 to 50 C. Cool to 30-35 C and add all other ingredients and mix. Cool to room temperature. Fill in roll-on or spray delivery system packaging.

1. A zeolite compound of formula (I); 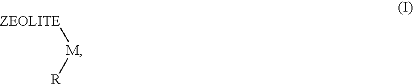
Wherein,
M is selected from Zn, Mn, Cu, Mo, Ti, Fe, Ni, Cr, Co, V, Ca, Ba, Mg, Se, Zr, and Al; and
R is selected from O-alkyl, O-aryl, O-heterocyclic, O-vinyl, O-Cycloalkyl, O-aralkyl, O-vinyl alkyl, O-vinyl aryl, O-vinyl heterocyclic, O-carboxy alkyl, O-carboxy aryl, O-keto alkyl, O-keto aryl, O-keto heterocyclic, O-keto alkyl amine, O-keto peptide, O-phosphate, S-alkyl, S-cycloalkyl, S-aralkyl, S-aryl, S-heterocyclic, S-vinyl, S-vinyl alkyl, S-vinyl aryl, S-vinyl heterocyclic, S-keto alkyl, S-keto aryl, S-keto heterocyclic, and S-keto alkyl amine.

2. A composition comprising the compound of claim 1 for the treatment of skin or hair condition.

3. A composition comprising the compound of claim 1, wherein said compound is zinc zeolite salicylate of formula (II);

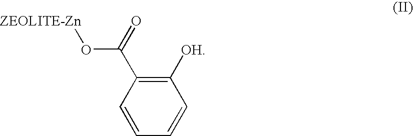

4. A composition comprising the compound of claim 1, wherein said compound is further selected from the group consisting of zinc zeolite salicylate, zinc zeolite lactate, (III); zinc zeolite ethylhexylglycerin, (IV); zinc zeolite benzoyl peroxide, (V); copper zeolite adenosine triphosphate, (VI); chromium zeolite benfotiamine, (VII); oxo vanadium zeolite pyridoxal-5-phosphate, (VII); and zinc zeolite triclosan (XIX):

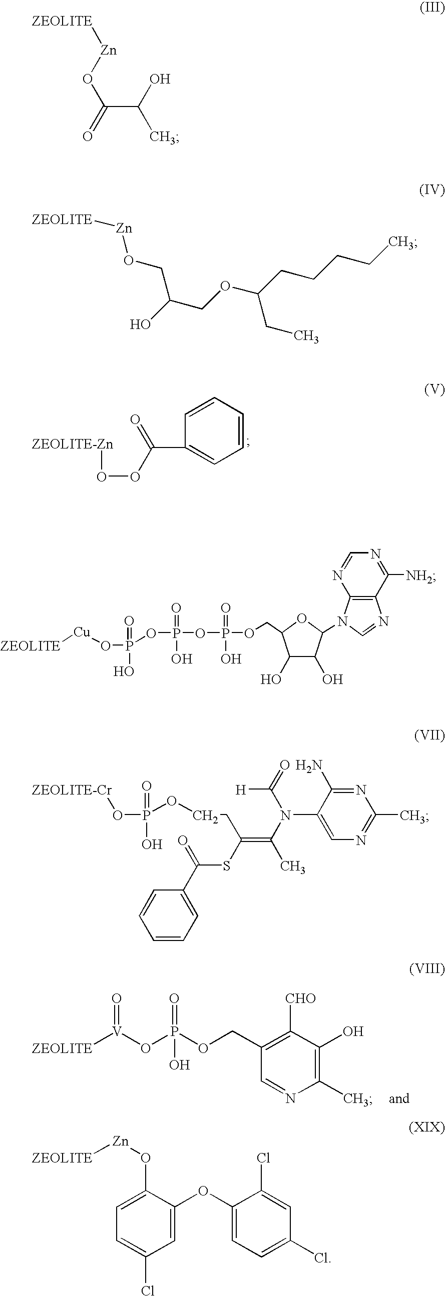

5. A composition comprising the compound of claim 1, wherein said compound is further selected from the group consisting of zirconium aluminum zeolite amino acid of formula (XX);
ZEOLITE-[Al]n.Zr(OH)₃n+4−x.Clx[AA]y, (XX);
Wherein,
n=3 to 10; and
x=3 to 9; and
AA=Amino Acid; and
y=0.05 to 3.

6. A composition according to claim 2, wherein said skin or hair condition is selected from the group consisting of skin aging including wrinkles and fine lines; darkened skin including age spots, dark circles around the eyes, and discoloration of skin from stretch marks; premature hair aging including hair loss and hair graying; acne including excess facial oil and facial pore size; loss of collagen including thinning skin and loss of skin pliability; body odor, including oral cavity odor, armpit odor, and incontinence odor; cellular inflammation including intracellular and extra cellular inflammation; malfunction of tyrosinase group of enzymes, malfunction of matrix metalloprotease group of enzymes; and combinations thereof.

7. A composition according to claim 4, wherein said compound is zinc zeolite salicylate.

8. A composition according to claim 4, wherein said compound is zinc zeolite Triclosan.

9. A composition according to claim 5, wherein said compound is zirconium aluminum zeolite glycinate; (XX): AA=glycine.

10. A composition according to claim 6, wherein said skin condition is darkened skin including age spots, dark circles around the eyes, and discoloration of skin from stretch marks.

11. A composition according to claim 6, wherein said hair condition is premature hair aging including hair loss and hair graying.

12. A method of topical delivery comprising a compound of claim 1 for the treatment of skin or hair condition; wherein,
(i) Said compound is applied topically; and, wherein,
(ii) Said application having been done either by a manual or a mechanical method, or a combination thereof; and, wherein,
(iii) Said application is repeated as necessary to treat said condition.

13. A method according to claim 12, and a base or carrier.

14. A method according to claim 12, wherein said skin or hair condition is selected from the group consisting of skin condition related to acne including excess facial oil and facial pore size; darkened skin including age spots, dark circles around the eyes, and discoloration of skin from stretch marks; skin aging including wrinkles and fine lines; loss of collagen including thinning skin and loss of skin pliability; cellular inflammation including intracellular and extra cellular inflammation; body odor, including oral cavity odor, armpit odor, and incontinence odor; premature hair aging including premature hair loss hair graying; malfunction of tyrosinase group of enzymes, malfunction of matrix metalloprotease group of enzymes; and combinations thereof.

15. A method according to claim 14, wherein said skin condition is darkened skin including age spots, dark circles around the eyes, and discoloration of skin from stretch marks.

16. A method according to claim 14, wherein said skin condition is skin aging including wrinkles and fine lines.

17. A method according to claim 14, wherein said hair condition is premature hair aging including premature hair loss and hair graying.

18. A method according to claim 14, wherein said skin condition is acne including excess facial oil and facial pore size.

19. A method according to claim 14, wherein said skin condition is body odor, including oral cavity odor, armpit odor, and incontinence odor.

20. A method according to claim 14, wherein said skin condition is loss of collagen including thinning skin and loss of skin pliability.
